SEQUENCE LISTING (27 sequences)

SEQ ID NO: 1 - protein, 14 aa, synthetic construct
  REGION 1..14: note = heavy chain CDR3H, <VEGF>ranibizumab; note = heavy chain CDR3H, <VEGF>ranibizumab
YPYYYGTSHWYFDV

SEQ ID NO: 2 - protein, 17 aa, synthetic construct
  REGION 1..17: note = heavy chain CDR2H, <VEGF>ranibizumab
WINTYTGEPTYAADFKR

SEQ ID NO: 3 - protein, 5 aa, synthetic construct
  REGION 1..5: note = heavy chain CDR1H, <VEGF>ranibizumab
HYGMN

SEQ ID NO: 4 - protein, 9 aa, synthetic construct
  REGION 1..9: note = light chain CDR3L, <VEGF>ranibizumab
QQYSTVPWT

SEQ ID NO: 5 - protein, 7 aa, synthetic construct
  REGION 1..7: note = light chain CDR2L, <VEGF>ranibizumab
FTSSLHS

SEQ ID NO: 6 - protein, 11 aa, synthetic construct
  REGION 1..11: note = light chain CDR1L, <VEGF>ranibizumab
SASQDISNYLN

SEQ ID NO: 7 - protein, 123 aa, synthetic construct [sequence omitted]

SEQ ID NO: 8 - protein, 107 aa, synthetic construct [sequence omitted]

SEQ ID NO: 9 - protein, 20 aa, synthetic construct
  REGION 1..20: note = heavy chain CDR3H, <ANG-2> Ang2i_LC10 variant
SPNPYYYDSSGYYYPGAFDI

SEQ ID NO: 10 - protein, 17 aa, synthetic construct
  REGION 1..17: note = heavy chain CDR2H, <ANG-2> Ang2i_LC10 variant
WINPNSGGTNYAQKFQG

SEQ ID NO: 11 - protein, 5 aa, synthetic construct
  REGION 1..5: note = heavy chain CDR1H, <ANG-2> Ang2i_LC10 variant
GYYMH

SEQ ID NO: 12 - protein, 11 aa, synthetic construct
  REGION 1..11: note = light chain CDR3L, <ANG-2> Ang2i_LC10 variant
QVWDSSSDHWV

SEQ ID NO: 13 - protein, 7 aa, synthetic construct
  REGION 1..7: note = light chain CDR2L, <ANG-2> Ang2i_LC10 variant
DDSDRPS

SEQ ID NO: 14 - protein, 11 aa, synthetic construct
  REGION 1..11: note = light chain CDR1L, <ANG-2> Ang2i_LC10 variant
GGNNIGSKSVH

SEQ ID NO: 15 - protein, 129 aa, synthetic construct
  REGION 1..129: note = heavy chain variable domain VH, <ANG-2> Ang2i_LC10 variant
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSS

SEQ ID NO: 16 - protein, 110 aa, synthetic construct
  REGION 1..110: note = light chain variable domain VL, <ANG-2> Ang2i_LC10 variant
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSS

SEQ ID NO: 17 - protein, 451 aa, synthetic construct
  REGION 1..451: note = Heavy chain 1 of <VEGF-ANG-2> CrossMAb IgG1 with AAA mutations and P329G LALA mutations (VEGFang2-0016)
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSP

SEQ ID NO: 18 - protein, 461 aa, synthetic construct
  REGION 1..461: note = Heavy chain 2 of <VEGF-ANG-2> CrossMAb IgG1 with AAA mutations and P329G LALA mutations (VEGFang2-0016)
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSP

SEQ ID NO: 19 - protein, 214 aa, synthetic construct
  REGION 1..214: note = Light chain 1 of <VEGF-ANG-2> CrossMAb IgG1 with AAA mutations and P329G LALA mutations (VEGFang2-0016)
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 213 aa, synthetic construct
  REGION 1..213: note = Light chain 2 of <VEGF-ANG-2> CrossMAb IgG1 with AAA mutations and P329G LALA mutations (VEGFang2-0016)
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 21 - protein, 107 aa, Homo sapiens [sequence omitted]

SEQ ID NO: 22 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 24 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 25 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 26 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 27 - protein, 1124 aa, Homo sapiens
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

(19) United States
(12) Patent Application Publication
(10) Pub. No.: US 2026/0365721 A1
(43) Pub. Date: Nov. 19, 2026

(54) TREATMENT OF AGE-RELATED MACULAR DEGENERATION AND DIABETIC MACULAR EDEMA BY ADMINISTRATION OF A BISPECIFIC ANTIBODY TO VEGF AND ANG-2

(71) Applicants: Hoffmann-La Roche Inc., Little Falls, NJ (US); Genentech, Inc., South San Francisco, CA (US)

(72) Inventors: Aaron Osborne, South San Francisco, CA (US); Jayashree Sahni, Basel (CH); Robert James Weikert, Basel (CH)

(73) Assignees: Hoffmann-La Roche Inc., Little Falls, NJ (US); Genentech, Inc., South San Francisco, CA (US)

(21) Appl. No.: 19/644,596

(22) Filed: Apr. 10, 2026

Related U.S. Application Data
(63) Continuation of application No. 18/166,996, filed on Feb. 9, 2023, now Pat. No. 12,624,098.
Application No. 18/166,996 is a continuation of application No. 16/985,477, filed on Aug. 5, 2020, now abandoned.
Application No. 16/985,477 is a continuation of application No. PCT/EP2019/052704, filed on Feb. 5, 2019.
(60) Provisional application No. 62/627,103, filed on Feb. 6, 2018.
(60) Provisional application No. 62/729,333, filed on Sep. 10, 2018.

(51) Int. Cl. C07K 16/22; A61K 39/00; A61P 27/02

(52) U.S. Cl. CPC C07K 16/22; A61P 27/02; A61K 2039/505; A61K 2039/545; C07K 2317/31; C07K 2317/565

(57) ABSTRACT

The current invention relates to the use of bispecific antibodies that bind to human vascular endothelial growth factor (VEGF) and to human angiopoietin-2 (ANG-2) for the treatment of ophthalmologic diseases, such as age-related macular degeneration and diabetic macular edema, where the bispecific antibodies are administered intravitreally every 8 weeks or less frequently.

### CROSS REFERENCE TO RELATED APPLICATIONS

This application is a continuation of U.S. patent application Ser. No. 18/166,996, filed Feb. 9, 2023 which is a continuation of U.S. patent application Ser. No. 16/985,477, filed Aug. 5, 2020, which is a continuation of International Application No. PCT/EP2019/052704, filed Feb. 5, 2019, claiming priority to U.S. provisional application No. 62/627,103 filed Feb. 6, 2018 and U.S. provisional application No. 62/729,333, filed Sep. 10, 2018, which are incorporated herein by reference in their entireties.

### SEQUENCE LISTING

This application contains a Sequence Listing which has been submitted electronically in XML format and is hereby incorporated by reference in its entirety. Said XML copy, created on Mar. 30, 2026 is named P34648-US-4_SEQ_LIST.xml and is 37,610 bytes in size.

The current invention relates to the use of antibodies which bind to VEGF and ANG2 for the treatment of ophthalmologic diseases.

### BACKGROUND OF THE INVENTION

Angiogenesis is implicated in the pathogenesis of a variety of disorders which include solid tumors, intraocular neovascular syndromes such as proliferative retinopathies or age-related macular degeneration (AMD), rheumatoid arthritis, and psoriasis (Folkman, J., et al., J. Biol. Chem. 267 (1992) 10931-10934; Klagsbrun, M., et al., Annu. Rev. Physiol. 53 (1991) 217-239; and Garner, A., Vascular diseases, in: Pathobiology of ocular disease, A dynamic approach, Garner, A., and Klintworth, G. K. (eds.), 2nd edition, Marcel Dekker, New York (1994), pp. 1625-1710).

Ranibizumab (trade name Lucentis®) is a monoclonal antibody fragment derived from the same parent murine antibody as bevacizumab (Avastin®). However, it has been affinity matured to provide stronger binding to VEGF-A (WO 98/45331). It is known that systemic blockade of VEGF-A is associated with an increased risk of certain adverse events, therefore ranibizumab is missing an Fc part in order to reduce systemic exposure and the risk of systemic toxicities. It is an anti-angiogenic agent that has been approved to treat the “wet” type of age-related macular degeneration (neovascular AMD), a common form of age-related vision loss.

Corneal angiogenesis assays have shown that both ANG-1 and ANG-2 had similar effects, acting synergistically with VEGF to promote growth of new blood vessels. Asahara, T., et al., Circ. Res. 83 (1998) 233-40. The possibility that there was a dose-dependent endothelial response was raised by the observation that in vitro at high concentration, ANG-2 can also be pro-angiogenic (Kim, I., et al., Oncogene 19 (2000) 4549-52). At high concentration, ANG-2 acts as an apoptosis survival factor for endothelial cells during serum deprivation apoptosis through activation of Tie2 via PI-3 Kinase and Akt pathway (Kim, I., et al., Oncogene 19 (2000) 4549-52).

Ocular vascular diseases such as “wet” age related macular degeneration (AMD) and proliferative diabetic retinopathy (PDR), are due to abnormal choroidal or retinal neovascularization respectively. Bleeding and leakage from these vessels can cause retinal dysfunction and loss of vision. Other retinal vascular disease, such as diabetic macular edema (DME) and macular edema secondary to retinal vein occlusion (RVO) are due to abnormal retinal leakage leading to retinal swelling and impairing visual function. These conditions are leading causes of visual loss in industrialized nations. Since the retina consists of well-defined layers of neuronal, glial, and vascular elements, relatively small disturbances such as those seen in vascular proliferation or edema can lead to significant loss of visual function. Inherited retinal degenerations, such as Retinitis Pigmentosa (RP), are also associated with vascular abnormalities, such as arteriolar narrowing and vascular atrophy. They affect as many as 1 in 3500 individuals and are characterized by progressive night blindness, visual field loss, optic nerve atrophy, arteriolar attenuation, and central loss of vision often progressing to complete blindness.

Ischemic retinopathies are characterized by loss or dysfunction of the retinal vasculature which results in a reduction of blood flow and hypoxia. The retina responds to hypoxia by generating signals to grow new blood vessels, but these new vessels are usually fragile and disorganized. It is the growth of these abnormal new vessels that creates most of the threat to vision since they can leak, hemorrhage or lead to scarring that may end in retinal detachment. Current treatments for ischemic retinopathies seek to halt the growth of the pathological vessels but do not address the underlying ischemia that drives their growth. Furthermore, standard treatment for diabetic retinopathy, an ischemic retinopathy that affects millions, involves destruction of a portion of the retina with a laser in an attempt destroy ischemic tissue in order to stop new vessel growth and preserve central vision. Strategies have been employed to block the function of vascular endothelial growth factor (VEGF), a major promoter of abnormal vessel growth and leakage. In the short term, anti-VEGF therapy can improve vision, but it does not address the underlying ischemia and in fact may exacerbate this condition as it inhibits all vessel growth, including beneficial collaterals. There is also the serious concern of systemic exposure of these drugs in elderly and/or diabetic patients where new vessel growth may be required in ischemic brains, hearts or limbs.

### SUMMARY OF THE INVENTION

According to one aspect of the present invention, methods, uses, bispecific antibodies (for use), medicaments or pharmaceutical formulations are provided for the treatment of patients suffering from an ocular vascular disease the method comprising administering to the patient an effective amount of a bispecific antibody which binds to human vascular endothelial growth factor (VEGF) and to human angiopoietin-2 (ANG-2),
wherein the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently in one embodiment every 16 weeks or less frequently).

One aspect of the invention is such method, use, bispecific antibody (for use), medicament or pharmaceutical formulation (for use) of/for treating a patient suffering from an ocular vascular disease the method, use, bispecific antibody (for use), medicament or pharmaceutical formulation (for use) comprising administering (intravitreally) to the patient an effective amount of a bispecific antibody which binds to human vascular endothelial growth factor (VEGF) and to human angiopoietin-2 (ANG-2), wherein the patient gains 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody. In one embodiment the bispecific antibody is administered intravitreally every 8 weeks or less frequently. One embodiment of the invention is a method of treating a patient suffering from an ocular vascular disease the method comprising administering (intravitreally) to the patient an effective amount of a bispecific antibody which binds to human vascular endothelial growth factor (VEGF) and to human angiopoietin-2 (ANG-2), wherein the patient experiences an improvement in vision subsequent to the administration of the bispecific VEGF/ANG2 antibody as measured by gaining 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody. In one embodiment the bispecific antibody is administered intravitreally every 8 weeks or less frequently.

In one embodiment of the invention the gain of letters in the BCVA/ETDRS letter score is measured at 4 weeks, and/or at 8 weeks, and/or at 12 weeks, and/or at 16 weeks, and/or at 20 weeks, and/or at 24 weeks after treatment start, respectively.

In one embodiment of the invention the gain of letters in the BCVA/ETDRS letter score is measured at 24 weeks, and/or at 25 weeks, and/or at 26 weeks, and/or at 27 weeks, and/or at 28 weeks, and/or at 29 weeks, and/or at 30 weeks, and/or at 31 weeks, and/or at 32 weeks, and/or at 33 weeks, and/or at 34 weeks, and/or at 35 weeks, and/or at 36 weeks, and/or at 37 weeks, and/or at 38 weeks, and/or at 39 weeks, and/or at 40 weeks, and/or at 41 weeks, and/or at 42 weeks, and/or at 43 weeks, and/or at 44 weeks, and/or at 45 weeks, and/or at 46 weeks, and/or at 47 weeks, and/or at 48 weeks, and/or at 49 weeks, and/or at 50 weeks, and/or at 51 weeks, and/or at 52 weeks, and/or at 53 weeks, and/or at 54 weeks, and/or at 55 weeks, and/or at 56 weeks, and/or at 57 weeks, and/or at 58 weeks, and/or at 59 weeks, and/or at 60 weeks after treatment start, respectively. In one embodiment of the invention the ocular vascular disease is selected from the group of: wet age-related macular degeneration (wet AMD), neovascular AMD, diabetic macular edema (DME), cystoid macular edema (CME), non-proliferative diabetic retinopathy (NPDR), proliferative diabetic retinopathy (PDR), macular edema secondary to central retinal vein occlusion, secondary to hemiretinal vein occlusion or secondary to branch vein occlusion, retinitis, conjunctivitis, uveitis, choroiditis, choroidal neovascularization (CNV) secondary to ocular inflammation including secondary to ocular histoplasmosis or presumed histoplasmosis or choroiditis; myopic choroidal neovascularization (mCNV). And choroidal neovascularization secondary to trauma, retinopathy of prematurity and rubeosis iridis/rubeotic glaucoma.

In one embodiment of the invention the ocular vascular disease is diabetic macular edema (DME).

In one embodiment of the invention the ocular vascular disease is diabetic macular edema (DME) and the gain of letters in the BCVA/ETDRS letter score is measured at about 9 to 15 month (in one embodiment at 9 to 14 month, in one embodiment at 9 to 12 month) after treatment start.

In one embodiment of the invention the ocular vascular disease is diabetic macular edema (DME) and the gain of letters in the BCVA/ETDRS letter score is measured at 36 weeks, and/or at 37 weeks, and/or at 38 weeks, and/or at 39 weeks, and/or at 40 weeks, and/or at 41 weeks, and/or at 42 weeks, and/or at 43 weeks, and/or at 44 weeks, and/or at 45 weeks, and/or at 46 weeks, and/or at 47 weeks, and/or at 48 weeks, and/or at 49 weeks, and/or at 50 weeks, and/or at 51 weeks, and/or at 52 weeks, and/or at 53 weeks, and/or at 54 weeks, and/or at 55 weeks, and/or at 56 weeks, and/or at 57 weeks, and/or at 58 weeks, and/or at 59 weeks, and/or at 60 weeks after treatment start, respectively.

These time points are quite early, typically maximum gains are not reached until about month 6-9 in nAMD and m 9-12 in DME

In one embodiment of the invention the ocular vascular disease is wet age-related macular degeneration (wet AMD) (,or neovascular age-related macular degeneration (nAMD).

In one embodiment of the invention the ocular vascular disease is wet age-related macular degeneration (wet AMD) (,or neovascular age-related macular degeneration (nAMD) and the gain of letters in the BCVA/ETDRS letter score is measured at about 9 to 15 month (in one embodiment at 6 to 9 month, in one embodiment at 6 to 12 month) after treatment start.

In one embodiment of the invention the ocular vascular disease is wet age-related macular degeneration (wet AMD) (,or neovascular age-related macular degeneration (nAMD) and the gain of letters in the BCVA/ETDRS letter score is measured at 24 weeks, and/or at 25 weeks, and/or at 26 weeks, and/or at 27 weeks, and/or at 28 weeks, and/or at 29 weeks, and/or at 30 weeks, and/or at 31 weeks, and/or at 32 weeks, and/or at 33 weeks, and/or at 34 weeks, and/or at 35 weeks, and/or at 36 weeks, and/or at 37 weeks, and/or at 38 weeks, and/or at 39 weeks, and/or at 40 weeks, and/or at 41 weeks, and/or at 42 weeks, and/or at 43 weeks, and/or at 44 weeks, and/or at 45 weeks, and/or at 46 weeks, and/or at 47 weeks, and/or at 48 weeks, and/or at 49 weeks, and/or at 50 weeks, and/or at 51 weeks, and/or at 52 weeks, and/or at 53 weeks, after treatment start, respectively.

In one embodiment of the invention the bispecific antibody which binds to human VEGF and to human ANG2 is a bispecific, bivalent anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat).

In one embodiment of the invention the patients suffering from an ocular vascular disease have not been previously treated with anti-VEGF treatment (e.g., monotherapy)(are treatment naïve).

In one embodiment of the invention the patients suffering from an ocular vascular disease have been previously treated with anti-VEGF treatment (e.g., monotherapy).

In one embodiment of the invention the ocular vascular disease is DME and the treatment of patients suffering from DME includes a fixed every 8^{th} week (Q8W) dosing schedule following treatment initiation.

In one embodiment of the invention the ocular vascular disease is DME and the treatment of patients suffering from DME includes a fixed Q12W dosing schedule following treatment initiation. In one embodiment of the invention following the treatment initiation, first one dose cycle of Q8W follows before the fixed Q12W dosing schedule.

In one embodiment of the invention the ocular vascular disease is DME and the treatment of patients suffering from DME includes following treatment initiation a dosing schedule that extends the administration interval in stable absence of disease, or shortens the interval if there is disease activity. In one embodiment of the invention such dosing schedule includes that the patient receives Q4W or Q8W or Q12W or Q16W dosing, dependent on their disease state. In one embodiment of the invention the stable absence of disease is determined as
Central Subfield Thickness (CST) increased by <50 μm; and/or
Best Corrected Visual Acuity (BCVA/ETDRS) decreased by <5 letters
and the disease activity is determined as
Central Subfield Thickness (CST) increased by ≥50 μm; and/or
Best Corrected Visual Acuity (BCVA/ETDRS) decreased by ≥5 letters.

In one embodiment of the invention the ocular vascular disease is AMD and the treatment of patients suffering from AMD includes following treatment initiation a dosing schedule that extends the administration interval in stable absence of disease, or shortens the interval if there is disease activity. In one embodiment of the invention such dosing schedule includes that the patient receives Q4W or Q8W or Q12W or Q16W dosing, dependent on their disease state. In one embodiment of the invention the stable absence of disease is determined as
Central Subfield Thickness (CST) increased by <50 μm; and/or
Best Corrected Visual Acuity (BCVA/ETDRS) decreased by <5 letters
and the disease activity is determined as
Central Subfield Thickness (CST) increased by ≥50 μm; and/or
Best Corrected Visual Acuity (BCVA/ETDRS) decreased by ≥5 letters.

### DESCRIPTION OF THE FIGURES

FIG. 1: BCVA change of DME patients treated from Baseline over Time to Week 24 (treatment naive patients). VA2 refers to the bispecific anti-VEGF/ANG2 antibody RO6867461 comprising the amino acid sequences of SEQ ID NO: 17, of SEQ ID NO: 18, of SEQ ID NO: 19, and of SEQ ID NO: 20 (administered intravitreally with a 6.0 mg or 1.5 mg dose), RBZ refers to ranibizumab (Lucentis®) ((administered intravitreally with a 0.3 mg dose))

FIG. 2: CST, central subfield thickness measured by SD OCT. CST change of DME patients treated from Baseline over Time to Week 24 (treatment naive patients). The bispecific anti-VEGF/ANG2 antibody RO6867461 comprising the amino acid sequences of SEQ ID NO: 17, of SEQ ID NO: 18, of SEQ ID NO: 19, and of SEQ ID NO: 20 (administered intravitreally with a 6.0 mg or 1.5 mg dose), was compared to ranibizumab (Lucentis®) ((administered intravitreally with a 0.3 mg dose)).

FIG. 3: Time to necessary retreatment based on disease activity assessed by both: BCVA decreased by ≥5 letters and CST increased by ≥50 μm (after dosing has discontinued (after 20 weeks or 6 monthly doses=Time post last intravitreal (IVT) administration). The bispecific anti-VEGF/ANG2 antibody RO6867461 comprising the amino acid sequences of SEQ ID NO: 17, of SEQ ID NO: 18, of SEQ ID NO: 19, and of SEQ ID NO: 20 (administered intravitreally with a 6.0 mg or 1.5 mg dose), was compared to ranibizumab (Lucentis®) ((administered intravitreally with a 0.3 mg dose)).

FIG. 4: Schematic comparison to other treatment options of DME based on published results (Compared agents Lucentis® (ranibizumab), Eylea® (aflibercept), brolucizumab and VA2 (RO6867461/RG7716).

FIG. 5: Overview of the study design for the evaluation of the bispecific antibody RO6867461 administered at 12- and 16-week intervals in patients with neovascular age-related macular degeneration (nAMD).

FIG. 6: BCVA gains from baseline of patients with neovascular age-related macular degeneration (nAMD) comparing the bispecific antibody RO6867461 (comprising the amino acid sequences of SEQ ID NO: 17, of SEQ ID NO: 18, of SEQ ID NO: 19, and of SEQ ID NO: 20 (administered intravitreally with a 6.0 mg) at 12- and 16-week intervals and ranibizumab (Lucentis®) ((administered intravitreally with a 0.3 mg dose)) at 4-week intervals.

FIG. 7: Change from baseline CST (measured via OCT) of patients with neovascular age-related macular degeneration (nAMD) comparing the bispecific antibody RO6867461 (comprising the amino acid sequences of SEQ ID NO: 17, of SEQ ID NO: 18, of SEQ ID NO: 19, and of SEQ ID NO: 20 (administered intravitreally with a 6.0 mg) at 12- and 16-week intervals and ranibizumab (Lucentis®) ((administered intravitreally with a 0.3 mg dose)) at 4-week intervals.

### DETAILED DESCRIPTION OF THE INVENTION

According to one aspect of the present invention, methods are provided for the treatment of patients suffering from an ocular vascular disease the method comprising administering to the patient an effective amount of a bispecific antibody which binds to human vascular endothelial growth factor (VEGF) and to human angiopoietin-2 (ANG-2),
wherein the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

One embodiment of the invention is a method of treating a patient suffering from a ocular vascular disease the method comprising administering to the patient an effective amount of a bispecific antibody which binds to human vascular endothelial growth factor (VEGF) and to human angiopoietin-2 (ANG-2), wherein the patient gains 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody. In one embodiment the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

One embodiment of the invention is a method of treating a patient suffering from a ocular vascular disease the method comprising administering to the patient an effective amount of a bispecific antibody which binds to human vascular endothelial growth factor (VEGF) and to human angiopoietin-2 (ANG-2), wherein the patient experiences an improvement in vision subsequent to the administration of the bispecific VEGF/ANG2 antibody as measured by gaining 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody. In one embodiment the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

In one embodiment of the invention the gain of letters in the BCVA/ETDRS letter score is measured at 4 weeks, and/or at 8 weeks, and/or at 12 weeks, and/or at 16 weeks, and/or at 20 weeks, and/or at 24 weeks after treatment start, respectively.

In one embodiment of the invention the gain of letters in the BCVA/ETDRS letter score is measured at 24 weeks, and/or at 25 weeks, and/or at 26 weeks, and/or at 27 weeks, and/or at 28 weeks, and/or at 29 weeks, and/or at 30 weeks, and/or at 31 weeks, and/or at 32 weeks, and/or at 33 weeks, and/or at 34 weeks, and/or at 35 weeks, and/or at 36 weeks, and/or at 37 weeks, and/or at 38 weeks, and/or at 39 weeks, and/or at 40 weeks, and/or at 41 weeks, and/or at 42 weeks, and/or at 43 weeks, and/or at 44 weeks, and/or at 45 weeks, and/or at 46 weeks, and/or at 47 weeks, and/or at 48 weeks, and/or at 49 weeks, and/or at 50 weeks, and/or at 51 weeks, and/or at 52 weeks, and/or at 53 weeks, and/or at 54 weeks, and/or at 55 weeks, and/or at 56 weeks, and/or at 57 weeks, and/or at 58 weeks, and/or at 59 weeks, and/or at 60 weeks after treatment start, respectively. In one embodiment of the invention the gain of letters in the BCVA/ETDRS letter score is measured at 45 weeks, and/or at 46 weeks, and/or at 47 weeks, and/or at 48 weeks, and/or at 49 weeks, and/or at 50 weeks, and/or at 51 weeks, and/or at 52 weeks, and/or at 53 weeks, and/or at 54 weeks, and/or at 55 weeks, and/or at 56 weeks, and/or at 57 weeks, and/or at 58 weeks, and/or at 59 weeks, and/or at 60 weeks after treatment start, respectively.

In one embodiment of the invention the method is used to prolong the time to retreatment and/or to prolong the time to loss of visual acuity and, wherein the retreatment with the bispecific antibody is administered in case of a disease activity which is determined as
Central Subfield Thickness (CST) increase by ≥50 μm (in one embodiment using spectral domain optical coherence tomography (SD-OCT)); and/or
Best Corrected Visual Acuity (BCVA/ETDRS) decrease by ≥5 letters.

One embodiment of the invention is a bispecific antibody which binds to human vascular endothelial growth factor (VEGF) and to human angiopoietin-2 (ANG-2), for use in the treatment of an ocular vascular disease,
wherein the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

One embodiment of the invention is a bispecific antibody which binds to human vascular endothelial growth factor (VEGF) and to human angiopoietin-2 (ANG-2), for use in the treatment of a patient suffering from an ocular vascular disease, wherein the patient gains 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody. In one embodiment the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

One embodiment of the invention is a bispecific antibody which binds to human vascular endothelial growth factor (VEGF) and to human angiopoietin-2 (ANG-2), for use in the treatment of a patient suffering from an ocular vascular disease, wherein the patient experiences an improvement in vision subsequent to the (intravitreal) administration of the bispecific VEGF/ANG2 antibody as measured by gaining 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody. In one embodiment the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

In one embodiment of the invention the gain of letters in the BCVA/ETDRS letter score is measured at 4 weeks, and/or at 8 weeks, and/or at 12 weeks, and/or at 16 weeks, and/or at 20 weeks, and/or at 24 weeks after treatment start, respectively.

In one embodiment of the invention the gain of letters in the BCVA/ETDRS letter score is measured at 45 weeks, and/or at 46 weeks, and/or at 47 weeks, and/or at 48 weeks, and/or at 49 weeks, and/or at 50 weeks, and/or at 51 weeks, and/or at 52 weeks, and/or at 53 weeks, and/or at 54 weeks, and/or at 55 weeks, and/or at 56 weeks, and/or at 57 weeks, and/or at 58 weeks, and/or at 59 weeks, and/or at 60 weeks after treatment start, respectively.

In one embodiment of the invention such bispecific antibody (for use) is used to prolong the time to retreatment and/or to prolong the time to loss of visual acuity and, wherein the retreatment with the bispecific antibody is administered in case of a disease activity which is determined as
Central Subfield Thickness (CST) increase by ≥50 μm (in one embodiment using spectral domain optical coherence tomography (SD-OCT)); and/or
Best Corrected Visual Acuity (BCVA/ETDRS) decrease by ≥5 letters.

One embodiment of the invention is a medicament or pharmaceutical formulation comprising a bispecific antibody which binds to human vascular endothelial growth factor (VEGF) and to human angiopoietin-2 (ANG-2), for use in the treatment of an ocular vascular disease,
wherein the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

One embodiment of the invention is a medicament or pharmaceutical formulation comprising a bispecific antibody which binds to human vascular endothelial growth factor (VEGF) and to human angiopoietin-2 (ANG-2), for use in the treatment of a patient suffering from an ocular vascular disease, wherein the patient gains 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody. In one embodiment the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

One embodiment of the invention is a medicament or pharmaceutical formulation comprising a bispecific antibody which binds to human vascular endothelial growth factor (VEGF) and to human angiopoietin-2 (ANG-2), for use in the treatment of a patient suffering from an ocular vascular disease, wherein the patient experiences an improvement in vision subsequent to the (intravitreal) administration of the bispecific VEGF/ANG2 antibody as measured by gaining 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody. In one embodiment the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

In one embodiment of the invention the gain of letters in the BCVA/ETDRS letter score is measured at 4 weeks, and/or at 8 weeks, and/or at 12 weeks, and/or at 16 weeks, and/or at 20 weeks, and/or at 24 weeks after treatment start, respectively.

In one embodiment of the invention the gain of letters in the BCVA/ETDRS letter score is measured at 45 weeks, and/or at 46 weeks, and/or at 47 weeks, and/or at 48 weeks, and/or at 49 weeks, and/or at 50 weeks, and/or at 51 weeks, and/or at 52 weeks, and/or at 53 weeks, and/or at 54 weeks, and/or at 55 weeks, and/or at 56 weeks, and/or at 57 weeks, and/or at 58 weeks, and/or at 59 weeks, and/or at 60 weeks after treatment start, respectively.

In one embodiment of the invention such medicament or pharmaceutical formulation is used to prolong the time to retreatment and/or to prolong the time to loss of visual acuity and, wherein the retreatment with the bispecific antibody is administered in case of a disease activity which is determined as
Central Subfield Thickness (CST) increase by ≥50 μm (in one embodiment using spectral domain optical coherence tomography (SD-OCT)); and/or
Best Corrected Visual Acuity (BCVA/ETDRS) decrease by ≥5 letters.

One embodiment of the invention is the use of a bispecific antibody which binds to human vascular endothelial growth factor (VEGF) and to human angiopoietin-2 (ANG-2), for the manufacture of a medicament for use in the treatment of an ocular vascular disease,
wherein the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

One embodiment of the invention is the use of a bispecific antibody which binds to human vascular endothelial growth factor (VEGF) and to human angiopoietin-2 (ANG-2), for the manufacture of a medicament for use in the treatment of an ocular vascular disease, wherein the patient gains 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody. In one embodiment the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

One embodiment of the invention is the use of a bispecific antibody which binds to human vascular endothelial growth factor (VEGF) and to human angiopoietin-2 (ANG-2), for the manufacture of a medicament for use in the treatment of an ocular vascular disease, wherein the patient experiences an improvement in vision subsequent to the (intravitreal) administration of the bispecific VEGF/ANG2 antibody as measured by gaining 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody. In one embodiment the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

In one embodiment of the invention the gain of letters in the BCVA/ETDRS letter score is measured at 4 weeks, and/or at 8 weeks, and/or at 12 weeks, and/or at 16 weeks, and/or at 20 weeks, and/or at 24 weeks after treatment start, respectively.

In one embodiment of the invention the gain of letters in the BCVA/ETDRS letter score is measured at 45 weeks, and/or at 46 weeks, and/or at 47 weeks, and/or at 48 weeks, and/or at 49 weeks, and/or at 50 weeks, and/or at 51 weeks, and/or at 52 weeks, and/or at 53 weeks, and/or at 54 weeks, and/or at 55 weeks, and/or at 56 weeks, and/or at 57 weeks, and/or at 58 weeks, and/or at 59 weeks, and/or at 60 weeks after treatment start, respectively.

In one embodiment of the invention medicament is used to prolong the time to retreatment and/or to prolong the time to loss of visual acuity and, wherein the retreatment with the bispecific antibody is administered in case of a disease activity which is determined as
Central Subfield Thickness (CST) increase by ≥50 μm (in one embodiment using spectral domain optical coherence tomography (SD-OCT)); and/or
Best Corrected Visual Acuity (BCVA/ETDRS) decrease by ≥5 letters.

In one embodiment BCVA determination in such method, use, bispecific antibody (for use), medicament or pharmaceutical formulation is based on the Early Treatment of Diabetic Retinopathy Study (ETDRS) Protocol adapted visual acuity charts and is assessed at a starting distance of 4 meters.

Such method, use, bispecific antibody (for use), medicament or pharmaceutical formulation may comprise sequentially administering initial doses (“treatment initiation”) (e.g. 3 to 7 monthly administrations; in one embodiment the treatment initiation includes 3 to 4 monthly administrations, in one embodiment the treatment initiation includes 4 to 5 monthly administrations; in one embodiment the treatment initiation includes 4 to 6 monthly administrations; in one embodiment the treatment initiation includes at least 4 monthly administrations; in one embodiment the treatment initiation includes 5 to 7 monthly administrations, in one embodiment the treatment initiation includes 6 monthly administrations) followed by one or more secondary doses of a therapeutically effective amount of the bispecific antibody, medicament or pharmaceutical formulation.

In one embodiment of the invention the bispecific antibody, medicament or pharmaceutical formulation is administered every 10 to 12 weeks (following treatment initiation).

In one embodiment of the invention the bispecific antibody, medicament or pharmaceutical formulation is administered every 11 to 13 weeks (following treatment initiation).

In one embodiment of the invention the bispecific antibody, medicament or pharmaceutical formulation is administered every 12 to 14 weeks (following treatment initiation).

In one embodiment of the invention the bispecific antibody, medicament or pharmaceutical formulation is administered every 13 to 15 weeks (following treatment initiation).

In one embodiment of the invention the bispecific antibody, medicament or pharmaceutical formulation is administered every 14 to 16 weeks (following treatment initiation).

In one embodiment of the invention the bispecific antibody, medicament or pharmaceutical formulation is administered every 10 to 11 weeks, or every 11 to 12 weeks, or every 12 to 13 weeks, or every 13 to 14 weeks, or every 14 to 15 weeks, or every 15 to 16 weeks (following treatment initiation, respectively).

In one embodiment of the invention the bispecific antibody, medicament or pharmaceutical formulation is administered every 10 weeks, or every 11 weeks, or every 12 weeks, or every 13 weeks, or every 14 weeks, or every 16 weeks (following treatment initiation, respectively).

In one embodiment of the invention the bispecific antibody, medicament or pharmaceutical formulation is administered in a dose of about 5 to 7 mg (at each treatment). In one embodiment the bispecific antibody is administered in a dose of 6 mg+/−10% (at each treatment). In one embodiment the bispecific antibody is administered in a dose of about 6 mg (at each treatment). (in one embodiment in a dose of 6 mg (at each treatment))

In one embodiment of the invention the bispecific antibody, medicament or pharmaceutical formulation is administered in a concentration of about 30 mg/ml of the bispecific antibody. In one embodiment of the invention the bispecific antibody, medicament or pharmaceutical formulation is administered in a concentration of about 120 mg/ml of the bispecific antibody.

The terms “ocular vascular disease” and “vascular eye disease” are used interchangeable herein and include, but are not limited to intraocular neovascular syndromes such as diabetic retinopathy, diabetic macular edema, retinopathy of prematurity, neovascular glaucoma, (branch) retinal vein occlusions, central retinal vein occlusions, macular degeneration, age-related macular degeneration, retinitis pigmentosa, retinal angiomatous proliferation, macular telangectasia, ischemic retinopathy, iris neovascularization, intraocular neovascularization, corneal neovascularization, retinal neovascularization, choroidal neovascularization, and retinal degeneration. (Garner, A., Vascular diseases, In: Pathobiology of ocular disease, A dynamic approach, Gamer, A., and Klintworth, G. K., (eds.), 2nd edition, Marcel Dekker, New York (1994), pp. 1625-1710). As used herein, ocular vascular disorder refers to any pathological conditions characterized by altered or unregulated proliferation and invasion of new blood vessels into the structures of ocular tissues such as the retina or cornea. In one embodiment the ocular vascular disease is selected from the group consisting of wet age-related macular degeneration (wet AMD), neovascular AMD (nAMD), diabetic macular edema (DME), cystoid macular edema (CME), non-proliferative diabetic retinopathy (NPDR), proliferative diabetic retinopathy (PDR), macular edema secondary to central retinal vein occlusion, secondary to hemiretinal vein occlusion or secondary to branch vein occlusion, retinitis, conjunctivitis, uveitis, choroiditis, choroidal neovascularization (CNV) secondary to ocular inflammation including secondary to ocular histoplasmosis or presumed histoplasmosis or choroiditis; myopic choroidal neovascularization (mCNV). And choroidal neovascularization secondary to trauma, retinopathy of prematurity and rubeosis iridis/rubeotic glaucoma, and other ophthalmic diseases wherein the eye disease or disorder is associated with ocular neovascularization, vascular leakage, and/or retinal edema. So the anti-VEGF/ANG2 bispecific antibodies for use and the methods described herein are useful in the prevention and treatment of wet AMD, nAMD CME, DME, NPDR, PDR, and uveitis, also preferably wet AMD, nAMD, also preferably DME, CME, NPDR and PDR, and also particularly wet AMD. In some embodiments, the ocular vascular disease is selected from the group consisting of wet age-related macular degeneration (wet AMD), neovascular age-related macular degeneration (nAMD), (diabetic) macular edema, retinal vein occlusions, retinopathy of prematurity, and diabetic retinopathy.

Other diseases/conditions associated with corneal neovascularization (or which may be the cause of corneal neovascularization) include, but are not limited to, epidemic keratoconjunctivitis, Vitamin A deficiency, contact lens overwear, atopic keratitis, superior limbic keratitis, pterygium keratitis sicca, sjogrens syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, rheumatoid arthritis, systemic lupus, polyarteritis, trauma, Wegeners sarcoidosis, Scleritis, Steven's Johnson disease, periphigoid radial keratotomy, and corneal graph rejection.

Diseases/conditions associated with retinal/choroidal neovascularization (or which may be the cause of retinal/choroidal neovascularization) include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, retinitis pigmentosa, retina edema (including macular edema), Eales disease, Bechets disease, infections causing a retinitis or choroiditis, presumed ocular histoplasmosis, Bests disease, myopia, optic (disc) pits, Stargardts disease, pars planitis, chronic retinal detachment, hyperviscosity syndromes, toxoplasmosis, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

Retinopathy of prematurity (ROP) is a disease of the eye that affects prematurely born babies. It is thought to be caused by disorganized growth of retinal blood vessels which may result in scarring and retinal detachment. ROP can be mild and may resolve spontaneously, but may lead to (total) blindness in serious cases. As such, all preterm babies are at risk for ROP, and very low birth weight is an additional risk factor. Both oxygen toxicity and relative hypoxia can contribute to the development of ROP.

Macular degeneration is a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. According to the American Academy of Ophthalmology, it is the leading cause of central vision loss (blindness) in the United States today for those over the age of fifty years. Although some macular dystrophies that affect younger individuals are sometimes referred to as macular degeneration, the term generally refers to age-related macular degeneration (AMD or ARMD).

“Age-related macular degeneration (AMD)”, as used herein, refers to a serious eye condition when the small central portion of the retina, known as the macula, deteriorates. AMD includes wet AMD and neovascular AMD. The wet form of AMD (wet AMD, wAMD or also called neovascular AMD, nAMD) is characterized by the growth of abnormal blood vessels from the choroid underneath the macula. This is called choroidal neovascularization. These blood vessels leak blood and fluid (below and) into the retina, causing (elevation of the retina and) distortion of vision that makes straight lines look wavy, as well as blind spots and loss of central vision. These abnormal blood vessels eventually scar, leading to permanent loss of central vision. The symptoms of AMD include dark, blurry areas in the center of vision; and diminished or changed color perception. AMD can be detected in a routine eye exam. One of the most common early signs of macular degeneration is the presence of drusen which are tiny yellow deposits under the retina and pigment clumping.

Advanced AMD, which is responsible for profound vision loss, has two forms: dry and wet. Central geographic atrophy, the dry form of advanced AMD, results from atrophy to the retinal pigment epithelial layer below the retina, which causes vision loss through loss of photoreceptors (rods and cones) in the central part of the eye. While no treatment is available for this condition, vitamin supplements with high doses of antioxidants, lutein and zeaxanthin, have been demonstrated by the National Eye Institute and others to slow the progression of dry macular degeneration and in some patients, improve visual acuity.

Retinitis pigmentosa (RP) is a group of genetic eye conditions. In the progression of symptoms for RP, night blindness generally precedes tunnel vision by years or even decades. Many people with RP do not become legally blind until their 40s or 50s and retain some sight all their life. Others go completely blind from RP, in some cases as early as childhood. Progression of RP is different in each case. RP is a type of hereditary retinal dystrophy, a group of inherited disorders in which abnormalities of the photoreceptors (rods and cones) or the retinal pigment epithelium (RPE) of the retina lead to progressive visual loss. Affected individuals first experience defective dark adaptation or nyctalopia (night blindness), followed by reduction of the peripheral visual field (known as tunnel vision) and, sometimes, loss of central vision late in the course of the disease.

Macular edema occurs when fluid and protein deposits collect on or under the macula of the eye, the central area of the retina responsible for fine vision, causing it to thicken and swell. The swelling may distort a person's central vision, as the macula is near the center of the retina at the back of the eyeball. This area holds tightly packed cones that provide sharp, clear central vision to enable a person to see form, color, and detail that is directly in the line of sight. Cystoid macular edema is a type of macular edema that includes cyst formation.

“Diabetic Macular Edema” (DME), as used herein, refers to a serious eye condition that affects people with diabetes (type 1 or 2). Macular edema occurs when blood vessels in the retina leak into the macula and fluid and protein deposits collect on or under the macula of the eye and causes it to thicken and swell (edema). The swelling may distort a person's central vision, as the macula is near the center of the retina at the back of the eyeball. The primary symptoms of DME include, but are not limited to, blurry vision, floaters, loss of contrast, double vision, and eventual loss of vision. The pathology of DME is characterized by breakdown of inner the blood-retinal barrier, normally preventing fluid movement in the retina, thus allowing fluid to accumulate in the retinal tissue, and presence of retinal thickening. DME is presently diagnosed during an eye examination consisting of a visual acuity test, which determines the smallest letters a person can read on a standardized chart, a dilated eye exam to check for signs of the disease, imaging tests such as optical coherence tomography (OCT) or fluorescein angiography (FA) and tonometry, an instrument that measures pressure inside the eye. The following studies are also performed to determine treatment: optical coherence tomography (OCT), fluorescein angiography, and color stereo fundus photography. DME can be broadly characterized into two main categories—Focal and Diffuse. Focal DME is characterized by specific areas of separate and distinct leakage in the macula with sufficient macular blood flow. Diffuse DME results from leakage of the entire capillary bed surrounding the macula, resulting from a breakdown of the inner blood-retina barrier of the eye. In addition to Focal and Diffuse, DME is also categorized based on clinical exam findings into clinically significant macular edema (CSME), non-CSME and CSME with central involvement (CSME-CI), which involves the fovea. The present invention includes methods to treat the above-mentioned categories of DME.

Best Corrected Visual Acuity (BCVA) is determined using methodology adapted from the 4-meter Early Treatment Diabetic Retinopathy Study [ETDRS] protocol (using Early Treatment Diabetic Retinopathy Study (ETDRS) like charts) and resulting in the respective letter score.

Disease activity is determined e.g. via reduction of the BCVA/ETDRs letter score and/or e.g. via the macular thickening by spectral domain optical coherence tomography (SD-OCT) involving the center of the macula as central subfield thickness (CST) (also known as center subfoveal thickness). In one preferred embodiment Central Subfield Thickness (CST) is determined using spectral domain optical coherence tomography (SD-OCT): In one preferred embodiment CST is measured by spectral domain optical coherence tomography (SD-OCT) with a Spectralis™ device; in one preferred embodiment CST is measured by spectral domain optical coherence tomography (SD-OCT) with a Cirrus™ device; in one embodiment CST is measured by spectral domain optical coherence tomography (SD-OCT) with a Topcon™ device; in one embodiment CST is measured by spectral domain optical coherence tomography (SD-OCT) with a Optovue™ device). As used herein, the term “a patient suffering from” refers to a human that exhibits one or more symptoms or indications of, and/or who has been diagnosed with an ocular vascular disease as described herein. The term “a patient suffering from” may also include, e.g., subjects who, prior to treatment, exhibit (or have exhibited) one or more indications of a vascular eye disease such as, e.g., retinal angiogenesis, neovascularization, vascular leak, retinal thickening of the center of the fovea, hard, yellow exudates of the center of the fovea with adjacent retinal thickening, and at least 1 disc area of retinal thickening, any part of which is within 1 disc diameter of the center of the fovea, blurry vision, floaters, loss of contrast, double vision, and eventual loss of vision.

As used herein, the term “a patient suffering from” may include a subset of population which is more susceptible to DME or AMD or may show an elevated level of a DME-associated or an AMD-associated biomarker. For example, “a subject in need thereof” may include a subject suffering from diabetes for more than 10 years, have frequent high blood sugar levels or high fasting blood glucose levels. In certain embodiments, the term “a patient suffering from” includes a subject who, prior to or at the time of administration of the bispecific anti-VEGF/ANG2 antibody, has or is diagnosed with diabetes. In certain embodiments, the term “a patient suffering from” includes a subject who, prior to or at the time of administration of the anti-VEGF/ANG2 antibody, is more than 50 years old. In some embodiments, the term “a patient suffering from” includes subjects who are smokers, or subjects with high blood pressure or high cholesterol.

The present invention includes methods or bispecific antibodies (for use), medicaments or pharmaceutical formulations for treating, preventing or reducing the severity of an ocular vascular disease comprising administering a therapeutically effective amount of a bispecific anti-VEGF/ANG2 antibody (or a medicament or pharmaceutical formulation comprising the bispecific anti-VEGF/ANG2 antibody) to a subject in need thereof, wherein the bispecific antibody, medicament or pharmaceutical formulation comprising such bispecific anti-VEGF/ANG2 antibody is administered (intravitreally) to the subject in multiple doses, e.g., as part of a specific therapeutic dosing regimen.

One embodiment of the invention is the method of treatment, use, bispecific antibody (for use), medicament or pharmaceutical formulation as described herein wherein patients suffering from an ocular vascular disease have not been previously treated with anti-VEGF treatment (e.g., monotherapy) (are treatment naïve).

One embodiment of the invention is the method of treatment, use, bispecific antibody (for use), medicament or pharmaceutical formulation as described herein wherein patients suffering from an ocular vascular disease have been previously treated with anti-VEGF treatment (e.g., monotherapy).

One embodiment of the invention is the method of treatment, use, bispecific antibody (for use), medicament or pharmaceutical formulation as described herein wherein the ocular vascular disease is DME and the treatment of patients suffering from DME includes a fixed every 8^{th} week (Q8W) dosing schedule following treatment initiation (In one embodiment the treatment initiation includes 5 to 7 monthly administrations; in one embodiment the treatment initiation includes 6 monthly administrations).

One embodiment of the invention is the method of treatment, use, bispecific antibody (for use), medicament or pharmaceutical formulation as described herein wherein the ocular vascular disease is DME and the treatment of patients suffering from DME includes a fixed Q12W dosing schedule following treatment initiation (In one embodiment the treatment initiation includes 5 to 7 monthly administrations; in one embodiment the treatment initiation includes 6 monthly administrations). In one embodiment following the treatment initiation, first one dose cycle of Q8W follows before the fixed Q12W dosing schedule.

One embodiment of the invention is the method of treatment, use, bispecific antibody (for use), medicament or pharmaceutical formulation as described herein wherein the ocular vascular disease is DME and the treatment of patients suffering from DME includes following treatment initiation a dosing schedule that extends the administration interval in stable absence of disease, or shortens the interval if there is disease activity (In one embodiment the treatment initiation includes 3 to 7 monthly administrations; in one embodiment the treatment initiation includes 3 to 5 monthly administrations; in one embodiment the treatment initiation includes at least 4 monthly administrations; in one embodiment the treatment initiation includes 4 to 6 monthly administrations). In one embodiment such dosing schedule includes that the patient receives Q4W or Q8W or Q12W or Q16W dosing, dependent on their disease state. In one embodiment the stable absence of disease is determined as
Central Subfield Thickness (CST) increased by <50 μm
Best Corrected Visual Acuity (BCVA/ETDRS) decreased by <5 letters
and the disease activity is determined as
Central Subfield Thickness (CST) increased by ≥50 μm
Best Corrected Visual Acuity (BCVA/ETDRS) decreased by ≥5 letters.

In one embodiment the stable absence of disease is determined as
Central Subfield Thickness (CST) is below about 300 μm (In one embodiment below 325 μm measured by spectral domain optical coherence tomography (SD-OCT) with a Spectralis™ device; in one embodiment below 315 μm measured by spectral domain optical coherence tomography (SD-OCT) with a Cirrus™ device; in one embodiment below 315 μm measured by spectral domain optical coherence tomography (SD-OCT) with a Topcon™ device; in one embodiment below 295 μm measured by spectral domain optical coherence tomography (SD-OCT) with a Optovue™ device),
and the disease activity is determined as
Central Subfield Thickness (CST) is above about 300 μm (In one embodiment above 325 μm measured by spectral domain optical coherence tomography (SD-OCT) with a Spectralis™ device; in one embodiment above 315 μm measured by spectral domain optical coherence tomography (SD-OCT) with a Cirrus™ device; in one embodiment above 315 μm measured by spectral domain optical coherence tomography (SD-OCT) with a Topcon™ device; in one embodiment above 295 μm measured by spectral domain optical coherence tomography (SD-OCT) with a Optovue™ device).

One embodiment of the invention the method of treatment, use, bispecific antibody (for use), medicament or pharmaceutical formulation as described herein wherein the ocular vascular disease is AMD (in one embodiment wet AMD) and the treatment of patients suffering from AMD (in one embodiment wet AMD) includes following treatment initiation a dosing schedule that extends the administration interval in stable absence of disease, or shortens the interval if there is disease activity (In one embodiment the treatment initiation includes 3 to 7 monthly administrations; in one embodiment the treatment initiation includes 3 to 5 monthly administrations; in one embodiment the treatment initiation includes at least 4 monthly administrations; in one embodiment the treatment initiation includes 4 to 6 monthly administrations). In one embodiment such dosing schedule includes that the patient receives Q4W or Q8W or Q12W or Q16W dosing, dependent on their disease state. In one embodiment the stable absence of disease is determined as
Central Subfield Thickness (CST) increased by <50 μm; and/or
Best Corrected Visual Acuity (BCVA/ETDRS) decreased by <5 letters and the disease activity is determined as
Central Subfield Thickness (CST) increased by ≥50 μm; and/or
Best Corrected Visual Acuity (BCVA/ETDRS) decreased by ≥5 letters.

In one embodiment the stable absence of disease is determined as
Central Subfield Thickness (CST) is below about 300 μm (In one embodiment below 325 μm measured by spectral domain optical coherence tomography (SD-OCT) with a Spectralis™ device; in one embodiment below 315 μm measured by spectral domain optical coherence tomography (SD-OCT) with a Cirrus™ device; in one embodiment below 315 μm measured by spectral domain optical coherence tomography (SD-OCT) with a Topcon™ device; in one embodiment below 295 μm measured by spectral domain optical coherence tomography (SD-OCT) with a Optovue™ device),
and the disease activity is determined as
Central Subfield Thickness (CST) is above about 300 μm (In one embodiment above 325 μm measured by spectral domain optical coherence tomography (SD-OCT) with a Spectralis™ device; in one embodiment above 315 μm measured by spectral domain optical coherence tomography (SD-OCT) with a Cirrus™ device; in one embodiment above 315 μm measured by spectral domain optical coherence tomography (SD-OCT) with a Topcon™ device; in one embodiment above 295 μm measured by spectral domain optical coherence tomography (SD-OCT) with a Optovue™ device).

In one embodiment the vascular ocular disease in such method, use, bispecific antibody (for use), medicament or pharmaceutical formulation is wetAMD (nAMD).

As used herein, “antibody” refers to a binding protein that comprises antigen-binding sites. The terms “binding site” or “antigen-binding site” as used herein denotes the region(s) of an antibody molecule to which a ligand actually binds. The term “antigen-binding site” comprises an antibody heavy chain variable domains (VH) and an antibody light chain variable domains (VL) (pair of VH/VL).).

Antibody specificity refers to selective recognition of the antibody for a particular epitope of an antigen. Natural antibodies, for example, are monospecific.

“Bispecific antibodies” according to the invention are antibodies which have two different antigen-binding specificities. Antibodies of the present invention are specific for two different antigens, VEGF as first antigen and ANG-2 as second antigen.

The term “monospecific” antibody as used herein denotes an antibody that has one or more binding sites each of which bind to the same epitope of the same antigen.

The term “valent” as used within the current application denotes the presence of a specified number of binding sites in an antibody molecule. As such, the terms “bivalent”, “tetravalent”, and “hexavalent” denote the presence of two binding site, four binding sites, and six binding sites, respectively, in an antibody molecule. The bispecific antibodies according to the invention are preferably “bivalent”.

The terms “bispecific antibody which binds to human vascular endothelial growth factor (VEGF) and to human angiopoietin-2 (ANG-2)”, “bispecific anti-VEGF/ANG2 antibody” and bispecific <VEGF/ANG2> antibody” as used herein are interchangeable and refer to an antibody which has at least two different antigen-binding sites, a first one which binds to VEGF and a second one which binds to ANG2.

Bispecific anti-VEGF/ANG2 antibodies are e.g. described in WO2010040508, WO2011/117329, WO2012/131078, WO2015/083978, WO2017/197199, and WO2014/009465. WO2014/009465 describes bispecific anti-VEGF/ANG2 antibodies especially designed for treatment of ocular vascular diseases. The bispecific anti-VEGF/ANG2 antibodies of WO2014/009465 (which is incorporated herein in its entirety) are especially useful in the treatment and treatment schedules of ocular vascular diseases as described herein.

In one embodiment the bispecific antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat).

In one embodiment such bispecific anti-VEGF/ANG2 antibody is bivalent.

In one embodiment such bispecific anti-VEGF/ANG2 antibody is characterized in that
i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

In one aspect of the invention such bispecific, bivalent antibody according to the invention is characterized in comprising
a) the heavy chain and the light chain of a first full length antibody that specifically binds to VEGF;
b) the modified heavy chain and modified light chain of a second full length antibody that specifically binds to ANG-2, wherein the constant domains CL and CH1 are replaced by each other.

This bispecific, bivalent antibody format for the bispecific antibody specifically binding to human vascular endothelial growth factor (VEGF) and human angiopoietin-2 (ANG-2) is described in WO 2009/080253 (including Knobs-into-Holes modified CH3 domains). The antibodies based on this bispecific, bivalent antibody format are named CrossMAbs.

In one embodiment such bispecific, bivalent anti-VEGF/ANG2 antibody is characterized in comprising
a) as heavy chain of the first full length antibody the amino acid sequence of SEQ ID NO: 17, and as light chain of the first full length antibody the amino acid sequence of SEQ ID NO: 18, and
b) as modified heavy chain of the second full length antibody the amino acid sequence of SEQ ID NO: 19, and as modified light chain of the second full length antibody the amino acid sequence of SEQ ID NO: 20.

In one embodiment such bispecific, bivalent anti-VEGF/ANG2 antibody is characterized in comprising the amino acid sequences of SEQ ID NO: 17, of SEQ ID NO: 18, of SEQ ID NO: 19, and of SEQ ID NO: 20. In one preferred embodiment the bispecific, bivalent anti-VEGF/ANG2 antibody is faricimab.

Accordingly, one embodiment of the invention is a bispecific, bivalent antibody comprising a first antigen-binding site that specifically binds to human VEGF and a second antigen-binding site that specifically binds to human ANG-2, characterized in comprising the amino acid sequences of SEQ ID NO: 17, of SEQ ID NO: 18, of SEQ ID NO: 19, and of SEQ ID NO: 20. In one preferred embodiment the bispecific, bivalent anti-VEGF/ANG2 antibody is faricimab.

In one embodiment the CH3 domains of the bispecific, bivalent antibody according to the invention is altered by the “knob-into-holes” technology which is described in detail with several examples in e.g. WO 96/027011, Ridgway J. B., et al., Protein Eng 9 (1996) 617-621; and Merchant, A. M., et al., Nat Biotechnol 16 (1998) 677-681. In this method the interaction surfaces of the two CH3 domains are altered to increase the heterodimerisation of both heavy chains containing these two CH3 domains. Each of the two CH3 domains (of the two heavy chains) can be the “knob”, while the other is the “hole”. The introduction of a disulfide bridge stabilizes the heterodimers (Merchant, A. M, et al., Nature Biotech 16 (1998) 677-681; Atwell, S., et al. J. Mol. Biol. 270 (1997) 26-35) and increases the yield.

In a preferred aspect of the invention the bispecific anti-VEGF/ANG2 antibodies according to the invention are characterized in that
the CH3 domain of one heavy chain and the CH3 domain of the other heavy chain each meet at an interface which comprises an original interface between the antibody CH3 domains;
wherein said interface is altered to promote the formation of the bispecific antibody, wherein the alteration is characterized in that:
a) the CH3 domain of one heavy chain is altered,
so that within the original interface the CH3 domain of one heavy chain that meets the original interface of the CH3 domain of the other heavy chain within the bispecific antibody,
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and
b) the CH3 domain of the other heavy chain is altered,
so that within the original interface of the second CH3 domain that meets the original interface of the first CH3 domain within the bispecific antibody
an amino acid residue is replaced with an amino acid residue having a smaller side chain volume, thereby generating a cavity within the interface of the second CH3 domain within which a protuberance within the interface of the first CH3 domain is positionable.

Thus the bispecific anti-VEGF/ANG2 antibodies for use described herein are preferably characterized in that
the CH3 domain of the heavy chain of the full length antibody of a) and the CH3 domain of the heavy chain of the full length antibody of b) each meet at an interface which comprises an alteration in the original interface between the antibody CH3 domains;
wherein i) in the CH3 domain of one heavy chain
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and wherein
ii) in the CH3 domain of the other heavy chain
an amino acid residue is replaced with an amino acid residue having a smaller side chain volume, thereby generating a cavity within the interface of the second CH3 domain within which a protuberance within the interface of the first CH3 domain is positionable.

Preferably said amino acid residue having a larger side chain volume is selected from the group consisting of arginine (R), phenylalanine (F), tyrosine (Y), tryptophan (W).

Preferably said amino acid residue having a smaller side chain volume is selected from the group consisting of alanine (A), serine (S), threonine (T), valine (V).

In one aspect of the invention both CH3 domains are further altered by the introduction of cysteine (C) as amino acid in the corresponding positions of each CH3 domain such that a disulfide bridge between both CH3 domains can be formed.

In one embodiment, the bispecific antibody comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M, et al., Nature Biotech 16 (1998) 677-681) e.g. by introducing a S354C mutation into one CH3 domain and a Y349C mutation into the other CH3 domain.

In a another preferred embodiment the bispecific antibody comprises S354C and T366W mutations in one of the two CH3 domains and Y349C, T366S, L368A, Y407V mutations in the other of the two CH3 domains In a another preferred embodiment the bispecific antibody comprises Y349C, T366W mutations in one of the two CH3 domains and S354C, T366S, L368A, Y407V mutations in the other of the two CH3 domains (the additional Y349C or S354C mutation in one CH3 domain and the additional S354C or Y349C mutation in the other CH3 domain forming a interchain disulfide bridge) (numbering always according to EU index of Kabat (Kabat, E. A., et al., Sequences of Proteins of Immunological Interest, 5th ed., Public Health Service, National Institutes of Health, Bethesda, MD (1991)).

Other techniques for CH3-modifications to enforce the heterodimerization are contemplated as alternatives of the invention and described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291.

In one embodiment the heterodimerization approach described in EP 1 870 459A1 is used alternatively. This approach is based on the introduction of substitutions/mutations of charged amino acids with the opposite charge at specific amino acid positions of the in the CH3/CH3 domain interface between both heavy chains. One preferred embodiment for said multispecific antibodies are amino acid R409D and K370E mutations in the CH3 domain of one heavy chain and amino acid D399K and E357K mutations in the CH3 domain of the other heavy chain of the multispecific antibody (numberings according to Kabat EU index).

In another embodiment said multispecific antibody comprises an amino acid T366W mutation in the CH3 domain of the “knobs chain” and amino acid T366S, L368A and Y407V mutations in the CH3 domain of the “hole chain”; and additionally comprises amino acid R409D and K370E mutations in the CH3 domain of the “knobs chain” and amino acid D399K and E357K mutations in the CH3 domain of the “hole chain”.

In one embodiment the heterodimerization approach described in WO2013/157953 is used alternatively. In one embodiment the CH3 domain of one heavy chain comprises an amino acid T366K mutation and the CH3 domain of the other heavy chain comprises an amino acid L351D mutation. In a further embodiment the CH3 domain of the one heavy chain further comprises an amino acid L351K mutation. In a further embodiment the CH3 domain of the other heavy chain further comprises an amino acid mutation selected from Y349E, Y349D and L368E (in one embodiment L368E).

In one embodiment the heterodimerization approach described in WO2012/058768 is used alternatively. In one embodiment the CH3 domain of one heavy chain comprises amino acid L351Y and Y407A mutations and the CH3 domain of the other heavy chain comprises amino acid T366A and K409F mutations. In a further embodiment the CH3 domain of the other heavy chain further comprises an amino acid mutation at position T411, D399, S400, F405, N390 or K392. In one embodiment said amino acid mutation is selected from the group consisting of
a) T411N, T411R, T411Q, T411K, T411D, T411E and T411W,
b) D399R, D399W, D399Y and D399K,
c) S400E, S400D, S400R and S400K,
d) F405I, F405M, F405T, F405S, F405V and F405W,
e) N390R, N390K and N390D,
f) K392V, K392M, K392R, K392L, K392F and K392E.

In a further embodiment the CH3 domain of one heavy chain comprises amino acid L351Y and Y407A mutations and the CH3 domain of the other heavy chain comprises amino acid T366V and K409F mutations. In a further embodiment the CH3 domain of one heavy chain comprises an amino acid Y407A mutation and the CH3 domain of the other heavy chain comprises amino acid T366A and K409F mutations. In a further embodiment the CH3 domain of the other heavy chain further comprises amino acid K392E, T411E, D399R and S400R mutations.

In one embodiment the heterodimerization approach described in WO2011/143545 is used alternatively. In one embodiment the amino acid modification according to WO2011/143545 is introduced in the CH3 domain of the heavy chain at a position selected from the group consisting of 368 and 409.

In one embodiment the heterodimerization approach described in WO2011/090762 which also uses the knob-into-hole technology described above is used alternatively. In one embodiment the CH3 domain of one heavy chain comprises an amino acid T366W mutation and the CH3 domain of the other heavy chain comprises an amino acid Y407A mutation. In one embodiment the CH3 domain of one heavy chain comprises an amino acid T366Y mutation and the CH3 domain of the other heavy chain comprises an amino acid Y407T mutation.

In one embodiment the multispecific antibody is of IgG2 isotype and the heterodimerization approach described in WO2010/129304 is used alternatively.

In one embodiment the heterodimerization approach described in WO2009/089004 is used alternatively. In one embodiment the CH3 domain of one heavy chain comprises an amino acid substitution of K392 or N392 with a negatively-charged amino acid (in one embodiment glutamic acid (E) or aspartic acid (D); in a further embodiment a K392D or N392D mutation) and the CH3 domain of the other heavy chain comprises an amino acid substitution of D399, E356, D356, or E357 with a positively-charged amino acid (in one embodiment Lysine (K) or arginine (R), in a further embodiment a D399K, E356K, D356K or E357K substitution; and in an even further embodiment a D399K or E356K mutation). In a further embodiment the CH3 domain of the one heavy chain further comprises an amino acid substitution of K409 or R409 with a negatively-charged amino acid (in one embodiment glutamic acid (E) or aspartic acid (D); in a further embodiment a K409D or R409D mutation). In a further embodiment the CH3 domain of the one heavy chain further or alternatively comprises an amino acid substitution of K439 and/or K370 with a negatively-charged amino acid (in one embodiment glutamic acid (E) or aspartic acid (D)).

In one embodiment the heterodimerization approach described in WO2007/147901 is used alternatively. In one embodiment the CH3 domain of one heavy chain comprises amino acid K253E, D282K and K322D mutations and the CH3 domain of the other heavy chain comprises amino acid D239K, E240K and K292D mutations.

In one embodiment the heterodimerization approach described in WO2007/110205 is used alternatively.

In one embodiment the bispecific antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat; and wherein
iv) in the constant heavy chain region a T366W mutation is comprised in one CH3 domain and T366S, L368A, Y407V mutations are comprised the other CH3 domain (numberings according to EU Index of Kabat).

In one embodiment the bispecific antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat; and wherein
iv) in the constant heavy chain region a S354C and T366W mutations are comprised in one CH3 domain and Y349C, T366S, L368A and Y407V mutations are comprised the other CH3 domain (numberings according to EU Index of Kabat).

In one embodiment such bispecific anti-VEGF/ANG2 antibody is bivalent.

In one embodiment such bispecific anti-VEGF/ANG2 antibody is characterized in that
i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

In one aspect of the invention such bispecific, bivalent antibody according to the invention is characterized in comprising
a) the heavy chain and the light chain of a first full length antibody that specifically binds to VEGF;
b) the modified heavy chain and modified light chain of a second full length antibody that specifically binds to ANG-2, wherein the constant domains CL and CH1 are replaced by each other.

The term “VEGF” as used herein refers to human vascular endothelial growth factor (VEGF/VEGF-A,) the 165-amino acid human vascular endothelial cell growth factor (amino acid 27-191 of precursor sequence of human VEGF165: SEQ ID NO: 24; amino acids 1-26 represent the signal peptide), and related 121, 189, and 206 vascular endothelial cell growth factor isoforms, as described by Leung, D. W., et al., Science 246 (1989) 1306-9; Houck et al., Mol. Endocrin. 5 (1991) 1806-1814; Keck, P. J., et al., Science 246 (1989) 1309-12 and Connolly, D. T., et al., J. Biol. Chem. 264 (1989) 20017-24; together with the naturally occurring allelic and processed forms of those growth factors. VEGF is involved in the regulation of normal and abnormal angiogenesis and neovascularization associated with tumors and intraocular disorders (Ferrara, N., et al., Endocr. Rev. 18 (1997) 4-25; Berkman, R. A., et al., J. Clin. Invest. 91 (1993) 153-159; Brown, L. F., et al., Human Pathol. 26 (1995) 86-91; Brown, L. F., et al., Cancer Res. 53 (1993) 4727-4735; Mattern, J., et al., Brit. J. Cancer. 73 (1996) 931-934; and Dvorak, H. F., et al., Am. J. Pathol. 146 (1995) 1029-1039). VEGF is a homodimeric glycoprotein that has been isolated from several sources and includes several isoforms. VEGF shows highly specific mitogenic activity for endothelial cells. A VEGF antagonist/inhibitor inhibits binding of VEGF to its receptor VEGFR. Known VEGF antagonist/inhibitors include bispecific anti-VEGF/ANG2 antibodies as described in WO2014/009465.

The term “ANG-2” as used herein refers to human angiopoietin-2 (ANG-2) (alternatively abbreviated with ANGPT2 or ANG2) (SEQ ID NO: 25) which is described e.g. in Maisonpierre, P. C., et al, Science 277 (1997) 55-60 and Cheung, A. H., et al., Genomics 48 (1998) 389-91. The angiopoietins-1 (SEQ ID NO: 26) and -2 were discovered as ligands for the Ties, a family of tyrosine kinases that is selectively expressed within the vascular endothelium (Yancopoulos, G. D., et al., Nature 407 (2000) 242-48). There are now four definitive members of the angiopoietin family. Angiopoietin-3 and -4 (Ang-3 and Ang-4) may represent widely diverged counterparts of the same gene locus in mouse and man (Kim, I., et al., FEBS Let, 443 (1999) 353-56; Kim, I., et al., J Biol Chem 274 (1999) 26523-28). ANG-1 and ANG-2 were originally identified in tissue culture experiments as agonist and antagonist, respectively (see for ANG-1: Davis, S., et al., Cell 87 (1996) 1161-69; and for ANG-2: Maisonpierre, P. C., et al., Science 277 (1997) 55-60). All of the known angiopoietins bind primarily to its receptor TIE2 (SEQ ID NO: 27), and both Ang-1 and -2 bind to TIE2 with an affinity of 3 nM (Kd) (Maisonpierre, P. C., et al., Science 277 (1997) 55-60). An ANG2 antagonist/inhibitor inhibits binding of ANG2 to its receptor TIE2. Known ANG2 antagonist/inhibitors include bispecific anti-VEGF/ANG2 antibodies as described in WO2014/009465.

An antigen-binding sites of the bispecific antibody of the invention contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences.

The antibodies of the invention comprise immunoglobulin constant regions derived from human origin of one or more immunoglobulin classes, wherein such immunoglobulin classes include IgG, IgM, IgA, IgD, and IgE classes and, in the case of IgG and IgA, their subclasses, especially IgG1 and IgG4.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties according to the invention, especially in regard to C1q binding and/or Fc receptor (FcR) binding. Such chimeric antibodies are also referred to as “class-switched antibodies.”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See, e.g., Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric antibodies. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Brueggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole, A., et al. and Boerner, P., et al. are also available for the preparation of human monoclonal antibodies (Cole, A., et al., Monoclonal Antibodies and Cancer Therapy, Liss, A. L., p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation).

The term “recombinant antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant antibodies have variable and constant regions in a rearranged form. The recombinant antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The “variable domain” (variable domain of a light chain (VL), variable domain of a heavy chain (VH) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies according to the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. CDRs on each chain are separated by such framework amino acids. Especially, CDR3 of the heavy chain is the region which contributes most to antigen binding. CDR and FR regions are determined according to the standard definition of Kabat, E. A., et al., Sequences of Proteins of Immunological Interest, 5th ed., Public Health Service, National Institutes of Health, Bethesda, MD (1991).

The term “full length antibody” denotes an antibody consisting of two “full length antibody heavy chains” and two “full length antibody light chains”. A “full length antibody heavy chain” is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. A “full length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be κ (kappa) or λ (lambda). The two full length antibody chains are linked together via inter-polypeptide disulfide bonds between the CL domain and the CH1 domain and between the hinge regions of the full length antibody heavy chains. Examples of typical full length antibodies are natural antibodies like IgG (e.g. IgG 1 and IgG2), IgM, IgA, IgD, and IgE. The full length antibodies according to the invention can be from a single species e.g. human, or they can be chimerized or humanized antibodies. The full length antibodies according to the invention comprise two antigen binding sites each formed by a pair of VH and VL, which both specifically bind to the same antigen. The C-terminus of the heavy or light chain of said full length antibody denotes the last amino acid at the C-terminus of said heavy or light chain. The N-terminus of the heavy or light chain of said full length antibody denotes the last amino acid at the N-terminus of said heavy or light chain.

The term “constant region” as used within the current applications denotes the sum of the domains of an antibody other than the variable region. The constant region is not involved directly in binding of an antigen, but exhibits various effector functions. Depending on the amino acid sequence of the constant region of their heavy chains, antibodies are divided in the classes: IgA, IgD, IgE, IgG and IgM, and several of these may be further divided into subclasses, such as IgG1, IgG2, IgG3, and IgG4, IgA1 and IgA2. The heavy chain constant regions that correspond to the different classes of antibodies are called α, δ, ε, γ, and μ, respectively. The light chain constant regions which can be found in all five antibody classes are called κ (kappa) and λ (lambda).

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Within the application for the numbering of positions and mutations the EU numbering system (EU Index) according to Kabat, E. A., et al., Sequences of Proteins of Immunological Interest, 5th ed., Public Health Service, National Institutes of Health, Bethesda, MD (1991) is used and referred to as “numbering according to EU Index of Kabat”.

In one embodiment the bispecific antibodies according to the invention have a constant region of human IgG1 subclass (derived from human IgG1 subclass). However, the C-terminal lysine (Lys447), or the C-terminal glycine (Gly446) and the C-terminal lysine (Lys447), of the Fc region may or may not be present.

In one embodiment the bispecific antibody as described herein is of IgG1 isotype/subclass and comprises a constant heavy chain domain of SEQ ID NO: 23 or the constant parts of the heavy chain amino acid sequence of SEQ ID NO: 17 and of the heavy chain amino acid sequence of SEQ ID NO: 18. In one embodiment additionally the C-terminal glycine (Gly446) is present. In one embodiment additionally the C-terminal glycine (Gly446) and the C-terminal lysine (Lys447) is present.

Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

In one embodiment the bispecific antibody according to the invention is of human IgG1 subclass with mutations L234A (Leu235Ala), L235A (Leu234Ala) and P329G (Pro329Gly). Such antibody has a reduced FcR binding (especially they show no more binding to FcRgammaI, FcRgammaII and FcRgammaIII). This especially useful to reduce potential side effects like e.g. thrombosis (Meyer, T., et al., J. Thromb. Haemost. 7 (2009) 171-81).

While Pro329Ala mutation which was described already removes only two third of the FcgammaRIIIa sandwich interaction, the Pro329Gly in the antibodies according to the invention fully imparts binding of the Fc part to FcgammaRIII. This is especially useful as the binding to FcgammaRIII is involved in ADCC (antibody-dependent cellular toxicity) which leads to cell death, which may be helpful in the treatment of cancer diseases, but which can cause serious side effect in the antibody based treatment of other vascular or immunological diseases. So the antibodies according to the invention of IgG1 subclass with mutations L234A, L235A and P329G and IgG4 subclass with mutations S228P, L235E and P329G are especially useful, as they both show no more binding to FcRgammaI, FcRgammaII and FcRgammaIII.

An “effective amount” of an agent, e.g., a pharmaceutical formulation or bispecific anti-VEGF/ANG2 antibody, refers to an amount effective, at dosages and for periods of time necessary, to achieve the desired therapeutic or prophylactic result.

In one embodiment of the invention the bispecific antibody, medicament or pharmaceutical formulation as described herein is administered via intravitreal application, e.g. via intravitreal injection (is administered “intravitreally”). This can be performed in accordance with standard procedures known in the art. See, e.g., Ritter et al., J. Clin. Invest. 116 (2006) 3266-76; Russelakis-Cameiro et al., Neuropathol. Appl. Neurobiol. 25 (1999) 196-206; and Wray et al., Arch. Neurol. 33 (1976) 183-5.

In some embodiments, therapeutic kits of the invention can contain one or more doses of the bispecific antibody described present in a medicament or pharmaceutical formulation, a suitable device for intravitreal injection of the medicament or pharmaceutical formulation, and an instruction detailing suitable subjects and protocols for carrying out the injection. In these embodiments, the medicament or pharmaceutical formulation are typically administered to the subject in need of treatment via intravitreal injection. This can be performed in accordance with standard procedures known in the art. See, e.g., Ritter et al., J. Clin. Invest. 116 (2006) 3266-76; Russelakis-Cameiro et al., Neuropathol. Appl. Neurobiol. 25 (1999) 196-206; and Wray et al., Arch. Neurol. 33 (1976) 183-5.

Regardless of the route of administration selected, the bispecific antibody as described herein is formulated into pharmaceutically acceptable dosage forms by conventional methods known to those of skill in the art.

Description of the amino acid sequences

| | | |
|---|---|---|
| SEQ ID NO: | 1 | heavy chain CDR3H, <VEGF>ranibizumab |
| SEQ ID NO: | 2 | heavy chain CDR2H, <VEGF>ranibizumab |
| SEQ ID NO: | 3 | heavy chain CDR1H, <VEGF>ranibizumab |
| SEQ ID NO: | 4 | light chain CDR3L, <VEGF>ranibizumab |
| SEQ ID NO: | 5 | light chain CDR2L, <VEGF>ranibizumab |
| SEQ ID NO: | 6 | light chain CDR1L, <VEGF>ranibizumab |
| SEQ ID NO: | 7 | heavy chain variable domain VH, |
| | | <VEGF>ranibizumab |
| SEQ ID NO: | 8 | light chain variable domain VL, |
| | | <VEGF>ranibizumab |
| SEQ ID NO: | 9 | heavy chain CDR3H, <ANG-2> Ang2i_LC10 variant |
| SEQ ID NO: | 10 | heavy chain CDR2H, <ANG-2> Ang2i_LC10 variant |
| SEQ ID NO: | 11 | heavy chain CDR1H, <ANG-2> Ang2i_LC10 variant |
| SEQ ID NO: | 12 | light chain CDR3L, <ANG-2> Ang2i_LC10 variant |
| SEQ ID NO: | 13 | light chain CDR2L, <ANG-2> Ang2i_LC10 variant |
| SEQ ID NO: | 14 | light chain CDR1L, <ANG-2> Ang2i_LC10 variant |
| SEQ ID NO: | 15 | heavy chain variable domain VH, <ANG-2> |
| | | Ang2i_LC10 variant |
| SEQ ID NO: | 16 | light chain variable domain VL, <ANG-2> |
| | | Ang2i_LC10 variant |
| SEQ ID NO: | 17 | Heavy chain 1 of <VEGF-ANG-2> CrossMAb |
| | | IgG1 with AAA mutations and P329G LALA |
| | | mutations (VEGFang2-0016) |
| SEQ ID NO: | 18 | Heavy chain 2 of <VEGF-ANG-2> CrossMAb IgG1 |
| | | with AAA mutations and P329G LALA mutations |
| | | (VEGFang2-0016) |
| SEQ ID NO: | 19 | Light chain 1 of <VEGF-ANG-2> CrossMAb IgG1 |
| | | with AAA mutations and P329G LALA mutations |
| | | (VEGFang2-0016) |
| SEQ ID NO: | 20 | Light chain 2 of <VEGF-ANG-2> CrossMAb IgG1 |
| | | with AAA mutations and P329G LALA mutations |
| | | (VEGFang2-0016) |
| SEQ ID NO: | 21 | kappa light chain constant region |
| SEQ ID NO: | 22 | lambda light chain constant region |
| SEQ ID NO: | 23 | heavy chain constant region derived from human |
| | | IgG1 |
| SEQ ID NO: | 24 | Human vascular endothelial growth factor (VEGF); |
| | | precursor sequence of human VEGF165 |
| SEQ ID NO: | 25 | Human angiopoietin-2 (ANG-2) |
| SEQ ID NO: | 26 | Human angiopoietin-1 (ANG-1) |
| SEQ ID NO: | 27 | Human Tie-2 receptor |

In the following, embodiments of the invention are listed:

1. A bispecific antibody which binds to human vascular endothelial growth factor (VEGF) and to human angiopoietin-2 (ANG-2), for use in the treatment of an ocular vascular disease,
wherein the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

2A. A bispecific antibody which binds to human vascular endothelial growth factor (VEGF) and to human angiopoietin-2 (ANG-2), for use in the treatment of a patient suffering from an ocular vascular disease, wherein the patient gains 12 or more letters (in one embodiment 13 or more letters, in one embodiment 14 or more letters, 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody.

2B. A bispecific antibody which binds to human vascular endothelial growth factor (VEGF) and to human angiopoietin-2 (ANG-2), for use in the treatment of a patient suffering from an ocular vascular disease, wherein the patient experiences an improvement in vision subsequent to the administration of the bispecific VEGF/ANG2 antibody as measured by gaining 12 or more letters (in one embodiment 13 or more letters, in one embodiment 14 or more letters, 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody.

3. The bispecific antibody (for use) according to any one of embodiments 2A to 2B,
wherein the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

4. The bispecific antibody (for use) according to any one of embodiments 1 to 3, wherein the gain of letters in the BCVA BCVA/ETDRS is measured at 4 weeks, and/or at 8 weeks, and/or at 12 weeks, and/or at 16 weeks, and/or at 20 weeks, and/or at 24 weeks after treatment start, respectively.

5 The bispecific antibody (for use) according to any one of embodiments 1 to 3, wherein the gain of letters in the BCVA BCVA/ETDRS is measured at 45 weeks, and/or at 46 weeks, and/or at 47 weeks, and/or at 48 weeks, and/or at 49 weeks, and/or at 50 weeks, and/or at 51 weeks, and/or at 52 weeks, and/or at 53 weeks, and/or at 54 weeks, and/or at 55 weeks, and/or at 56 weeks, and/or at 57 weeks, and/or at 58 weeks, and/or at 59 weeks, and/or at 60 weeks after treatment start, respectively.

6. The bispecific antibody (for use) according to any one of embodiments 1 to 5, wherein the bispecific antibody is used to prolong the time to retreatment and/or to prolong the time to loss of visual acuity (e.g. Best Corrected Visual Acuity (BCVA) BCVA/ETDRS) and, wherein the retreatment is deemed necessary in case of disease activity which is determined as
Central Subfield Thickness (CST) increased by ≥50 μm (in one embodiment using spectral domain optical coherence tomography (SD-OCT)); and/or
Best Corrected Visual Acuity (BCVA/ETDRS) decreased by ≥5 letters.

7. The bispecific antibody (for use) according to any one of embodiments 1 to 6 wherein the bispecific antibody is administered following a treatment initiation of 3 to 7 monthly administrations (in one embodiment the treatment initiation includes 3 to 5 monthly administrations, in one embodiment the treatment initiation includes 4 monthly administrations; in one embodiment the treatment initiation includes 5 to 7 monthly administrations, in one embodiment the treatment initiation includes 6 monthly administrations).

8. The bispecific antibody (for use) according to any one of embodiments 1 to 7, wherein the ocular vascular disease is selected from the group of: wet age-related macular degeneration (wet AMD), neovascular AMD, diabetic macular edema (DME), cystoid macular edema (CME), non-proliferative diabetic retinopathy (NPDR), proliferative diabetic retinopathy (PDR), macular edema secondary to central retinal vein occlusion, secondary to hemiretinal vein occlusion or secondary to branch vein occlusion, retinitis, conjunctivitis, uveitis, choroiditis, choroidal neovascularization (CNV) secondary to ocular inflammation including secondary to ocular histoplasmosis or presumed histoplasmosis or choroiditis; myopic choroidal neovascularization (mCNV). And choroidal neovascularization secondary to trauma, retinopathy of prematurity and rubeosis iridis/rubeotic glaucoma.

9. The bispecific antibody (for use) according to any one of embodiments 1 to 7 wherein the ocular vascular disease is diabetic macular edema (DME).

10. The bispecific antibody (for use) according to any one of embodiments 1 to 7, wherein the ocular vascular disease is wet age-related macular degeneration (wet AMD), or neovascular age-related macular degeneration (nAMD).

11. The bispecific antibody (for use) according to any one of embodiments 1 to 10, wherein the bispecific antibody which binds to VEGF and to human ANG-2 is a VEGF antagonist/inhibitor and an ANG2 antagonist/inhibitor or inhibits binding of VEGF to its receptor VEGFR and inhibits binding of ANG2 to its receptor TIE2.

12. The bispecific antibody (for use) according to any one of embodiments 1 to 11, wherein the bispecific antibody is administered every 10 to 12 weeks.

13. The bispecific antibody (for use) according to any one of embodiments 1 to 11, wherein the bispecific antibody is administered every 11 to 13 weeks.

14. The bispecific antibody (for use) according to any one of embodiments 1 to 11 wherein the bispecific antibody is administered every 12 to 14 weeks.

15. The bispecific antibody (for use) according to any one of embodiments 1 to 11 wherein the bispecific antibody is administered every 13 to 15 weeks.

16. The bispecific antibody (for use) according to any one of embodiments 1 to 11 wherein the bispecific antibody is administered every 14 to 16 weeks.

17. The bispecific antibody (for use) according to any one of embodiments 1 to 16, wherein the bispecific antibody which binds to human VEGF and to human ANG2 is a bispecific, bivalent anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14,
and wherein
iii) the bispecific antibody comprises a constant heavy chain region of human IgG1 subclass comprising the mutations I253A, H310A, and H435A and the mutations L234A, L235A and P329G (numberings according to EU Index of Kabat).

18. The bispecific antibody (for use) according to embodiment 17, wherein
i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

19. The bispecific antibody (for use) according to embodiment 18, wherein the bispecific antibody which binds to human VEGF and human ANG2 comprises the amino acid sequences of SEQ ID NO: 17, of SEQ ID NO: 18, of SEQ ID NO: 19, and of SEQ ID NO: 20.

20. The bispecific antibody (for use) according to any one of embodiments 17 to 19, wherein the bispecific antibody is administered in a dose of about 5 to 7 mg (at each treatment).

21. The bispecific antibody (for use) according to any one of embodiments 17 to 19, wherein the bispecific antibody is administered in a dose of about 6 mg (at each treatment) (in one embodiment in a dose of 6 mg+/−10% (at each treatment); (in one embodiment in a dose of 6 mg (at each treatment)))

22. The bispecific antibody (for use) according to any one of embodiments 20 to 21, wherein the bispecific antibody is administered at a concentration of about 30 mg/ml.

23. The bispecific antibody (for use) according to any one of embodiments 20 to 21, wherein the bispecific antibody is administered at a concentration of about 120 mg/ml.

24. The bispecific antibody (for use) according to any one of the preceding embodiments wherein patients suffering from an ocular vascular disease have not been previously treated with anti-VEGF treatment (e.g., monotherapy) (are treatment naïve).

25. The bispecific antibody (for use) according to any one of the preceding embodiments wherein patients suffering from an ocular vascular disease have been previously treated with anti-VEGF treatment (e.g., monotherapy).

26. The bispecific antibody (for use) according to the preceding embodiments wherein the ocular vascular disease is DME and the treatment of patients suffering from DME includes a fixed every 8^{th} week (Q8W) dosing schedule following treatment initiation (In one embodiment the treatment initiation includes 5 to 7 monthly administrations; in one embodiment the treatment initiation includes 6 monthly administrations).

27. The bispecific antibody (for use) according to the preceding embodiments wherein the ocular vascular disease is DME and the treatment of patients suffering from DME includes a fixed Q12W dosing schedule following treatment initiation (In one embodiment the treatment initiation includes 5 to 7 monthly administrations; in one embodiment the treatment initiation includes 6 monthly administrations).

28. The bispecific antibody (for use) according to embodiment 27 wherein, following the treatment initiation, first one dose cycle of Q8W follows before the fixed Q12W dosing schedule.

29. The bispecific antibody (for use) according to the preceding embodiments wherein the ocular vascular disease is DME and the treatment of patients suffering from DME includes following treatment initiation a dosing schedule that extends the administration interval in stable absence of disease, or shortens the interval if there is disease activity (In one embodiment the treatment initiation includes 3 to 7 monthly administrations; in one embodiment the treatment initiation includes 4 to 6 monthly administrations).

30. The bispecific antibody (for use) according to embodiment 29 wherein such dosing schedule includes that the patient receives Q8W or Q12W or Q16W dosing, dependent on their disease state (in one embodiment Q4W or Q8W or Q12W or Q16W dosing, dependent on their disease state)

31. The bispecific antibody (for use) according to embodiment 29 or 30, wherein the stable absence of disease is determined as
Central Subfield Thickness (CST) increased by <50 μm; and/or
Best Corrected Visual Acuity (BCVA/ETDRS) decreased by <5 letters
and the disease activity is determined as
Central Subfield Thickness (CST) increased by ≥50 μm; and/or
Best Corrected Visual Acuity (BCVA/ETDRS) decreased by ≥5 letters.

32. The method according to embodiment 29 or 30, wherein the stable absence of disease is determined as
Central Subfield Thickness (CST) is below about 300 μm (In one embodiment below 325 μm measured by spectral domain optical coherence tomography (SD-OCT) with a Spectralis™ device; in one embodiment below 315 μm measured by spectral domain optical coherence tomography (SD-OCT) with a Cirrus™ device; in one embodiment below 315 μm measured by spectral domain optical coherence tomography (SD-OCT) with a Topcon™ device; in one embodiment below 295 μm measured by spectral domain optical coherence tomography (SD-OCT) with a Optovue™ device),
and the disease activity is determined as
Central Subfield Thickness (CST) is above about 300 μm (In one embodiment above 325 μm measured by spectral domain optical coherence tomography (SD-OCT) with a Spectralis™ device; in one embodiment above 315 μm measured by spectral domain optical coherence tomography (SD-OCT) with a Cirrus™ device; in one embodiment above 315 μm measured by spectral domain optical coherence tomography (SD-OCT) with a Topcon™ device; in one embodiment above 295 μm measured by spectral domain optical coherence tomography (SD-OCT) with a Optovue™ device).

33. The bispecific antibody (for use) according to the preceding embodiments wherein the ocular vascular disease is AMD (in one embodiment wet AMD) and the treatment of patients suffering from AMD (in one embodiment wet AMD) includes following treatment initiation (In one embodiment the treatment initiation includes 3 to 7 monthly administrations; in one embodiment the treatment initiation includes 4 to 6 monthly administrations) a dosing schedule that extends the administration interval in stable absence of disease, or shortens the interval if there is disease activity.

34. The bispecific antibody (for use) according to embodiment 33 wherein such dosing schedule includes that the patient receives Q8W or Q12W or Q16W dosing, dependent on their disease state (in one embodiment Q4W or Q8W or Q12W or Q16W dosing, dependent on their disease state).

35. The bispecific antibody (for use) according to embodiment 33 or 34, wherein the stable absence of disease is determined as
Central Subfield Thickness (CST) increased by <50 μm; and/or
Best Corrected Visual Acuity (BCVA/ETDRS) decreased by <5 letters
and the disease activity is determined as
Central Subfield Thickness (CST) increased by ≥50 μm; and/or
Best Corrected Visual Acuity (BCVA/ETDRS) decreased by ≥5 letters.

36. The bispecific antibody (for use) according to embodiment 33 or 34, wherein the stable absence of disease is determined as
Central Subfield Thickness (CST) is below about 300 μm (In one embodiment below 325 μm measured by spectral domain optical coherence tomography (SD-OCT) with a Spectralis™ device; in one embodiment below 315 μm measured by spectral domain optical coherence tomography (SD-OCT) with a Cirrus™ device; in one embodiment below 315 μm measured by spectral domain optical coherence tomography (SD-OCT) with a Topcon™ device; in one embodiment below 295 μm measured by spectral domain optical coherence tomography (SD-OCT) with a Optovue™ device),
and the disease activity is determined as
Central Subfield Thickness (CST) is above about 300 μm (In one embodiment above 325 μm measured by spectral domain optical coherence tomography (SD-OCT) with a Spectralis™ device; in one embodiment above 315 μm measured by spectral domain optical coherence tomography (SD-OCT) with a Cirrus™ device; in one embodiment above 315 μm measured by spectral domain optical coherence tomography (SD-OCT) with a Topcon™ device; in one embodiment above 295 μm measured by spectral domain optical coherence tomography (SD-OCT) with a Optovue™ device).

In the following, embodiments of the invention are listed:

1. A method of treating a patient suffering from an ocular vascular disease the method comprising administering to the patient an effective amount of a bispecific antibody which binds to human vascular endothelial growth factor (VEGF) and to human angiopoietin-2 (ANG-2),
wherein the bispecific antibody is administered intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

2A. A method of treating a patient suffering from an ocular vascular disease the method comprising administering to the patient an effective amount of a bispecific antibody which binds to human vascular endothelial growth factor (VEGF) and to human angiopoietin-2 (ANG-2), wherein the patient gains 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody.

2B. A method of treating a patient suffering from a ocular vascular disease the method comprising administering to the patient an effective amount of a bispecific antibody which binds to human vascular endothelial growth factor (VEGF) and to human angiopoietin-2 (ANG-2), wherein the patient experiences an improvement in vision subsequent to the administration of the bispecific VEGF/ANG2 antibody as measured by gaining 12 or more letters (in one embodiment 13 or more letters, in one embodiment 14 or more letters, in one embodiment 15 or more letters) of Best Corrected Visual Acuity (BCVA) measured using Early Treatment Diabetic Retinopathy Study (ETDRS) like charts, compared to the patient's BCVA letter score prior to the dosing of the bispecific VEGF/ANG2 antibody.

3. The method according to any one of embodiments 2A to 2B,
wherein the bispecific antibody is administered (is to be administered) intravitreally every 8 weeks or less frequently (in one embodiment every 9 weeks or less frequently; in one embodiment every 10 weeks or less frequently; in one embodiment every 11 weeks or less frequently; in one embodiment every 12 weeks or less frequently; in one embodiment every 13 weeks or less frequently; in one embodiment every 14 weeks or less frequently; in one embodiment every 15 weeks or less frequently).

4. The method according to any one of embodiments 1 to 3, wherein the gain of letters in the BCVA/ETDRS letter score is measured at 4 weeks, and/or at 8 weeks, and/or at 12 weeks, and/or at 16 weeks, and/or at 20 weeks, and/or at 24 weeks after treatment start, respectively.

5. The method according to any one of embodiments 1 to 3, wherein the gain of letters in the BCVA/ETDRS letter score is measured at 45 weeks, and/or at 46 weeks, and/or at 47 weeks, and/or at 48 weeks, and/or at 49 weeks, and/or at 50 weeks, and/or at 51 weeks, and/or at 52 weeks, and/or at 53 weeks, and/or at 54 weeks, and/or at 55 weeks, and/or at 56 weeks, and/or at 57 weeks, and/or at 58 weeks, and/or at 59 weeks, and/or at 60 weeks after treatment start, respectively.

6. The method according to any one of embodiments 1 to 5, wherein the bispecific antibody is used to prolong the time to retreatment and/or to prolong the time to loss of visual acuity and, wherein the retreatment with the bispecific antibody is administered in case of a disease activity which is determined as
Central Subfield Thickness (CST) increase by ≥50 μm (in one embodiment using spectral domain optical coherence tomography (SD-OCT)); and/or
Best Corrected Visual Acuity (BCVA/ETDRS) decrease by ≥5 letters.

7. The method according to any one of embodiments 1 to 6, wherein the bispecific antibody is administered following a treatment initiation of 3 to 7 monthly administrations (in one embodiment the treatment initiation includes 3 to 5 monthly administrations, in one embodiment the treatment initiation includes 4 monthly administrations in one embodiment the treatment initiation includes 5 to 7 monthly administrations, in one embodiment the treatment initiation includes 6 monthly administrations).

8. The method according to any one of embodiments 1 to 7, wherein the ocular vascular disease is selected from the group of wet age-related macular degeneration (wet AMD), neovascular AMD, diabetic macular edema (DME), cystoid macular edema (CME), non-proliferative diabetic retinopathy (NPDR), proliferative diabetic retinopathy (PDR), macular edema secondary to central retinal vein occlusion, secondary to hemiretinal vein occlusion or secondary to branch vein occlusion, retinitis, conjunctivitis, uveitis, choroiditis, choroidal neovascularization (CNV) secondary to ocular inflammation including secondary to ocular histoplasmosis or presumed histoplasmosis or choroiditis; myopic choroidal neovascularization (mCNV). And choroidal neovascularization secondary to trauma, retinopathy of prematurity and rubeosis iridis/rubeotic glaucoma.

9. The method according to any one of embodiments 1 to 7, wherein the ocular vascular disease is diabetic macular edema (DME).

10. The method according to any one of embodiments 1 to 7, wherein the ocular vascular disease is wet age-related macular degeneration (wet AMD), or neovascular age-related macular degeneration (nAMD).

11. The method according to any one of embodiments 1 to 10, wherein the bispecific antibody which binds to VEGF and to human ANG-2 is a VEGF antagonist/inhibitor and an ANG2 antagonist/inhibitor or inhibits binding of VEGF to its receptor VEGFR and inhibits binding of ANG2 to its receptor TIE2.

12. The method according to any one of embodiments 1 to 11, wherein the bispecific antibody is administered every 10 to 12 weeks.

13. The method according to any one of embodiments 1 to 11, wherein the bispecific antibody is administered every 11 to 13 weeks

14. The method according to any one of embodiments 1 to 11, wherein the bispecific antibody is administered every 12 to 14 weeks.

15. The method according to any one of embodiments 1 to 11, wherein the bispecific antibody is administered every 13 to 15 weeks.

16. The method according to any one of embodiments 1 to 11, wherein the bispecific antibody is administered every 14 to 16 weeks.

17. The method according to any one of embodiments 1 to 16, wherein the bispecific antibody which binds to human VEGF and to human ANG2 is a bispecific, bivalent anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat).

18. The method according to embodiment 17, wherein
i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

19. The method according to embodiment 18, wherein the bispecific antibody which binds to human VEGF and human ANG2 comprises the amino acid sequences of SEQ ID NO: 17, of SEQ ID NO: 18, of SEQ ID NO: 19, and of SEQ ID NO: 20.

20. The method according to any one of embodiments 17 to 19, wherein the bispecific antibody is administered in a dose of about 5 to 7 mg (at each treatment).

21. The method according to any one of embodiments 17 to 19, wherein the bispecific antibody is administered in a dose of about 6 mg (at each treatment) (in one embodiment in a dose of 6 mg+/−10% (at each treatment) (in one embodiment in a dose of 6 mg (at each treatment))).

22. The method according to any one of embodiments 20 to 21, wherein the bispecific antibody is administered at a concentration of about 30 mg/ml.

23. The method according to any one of embodiments 20 to 21, wherein the bispecific antibody is administered at a concentration of about 120 mg/ml.

24. The method according to any one of the preceding embodiments wherein patients suffering from an ocular vascular disease have not been previously treated with anti-VEGF treatment (e.g., monotherapy) (are treatment naïve).

25. The method according to any one of the preceding embodiments wherein patients suffering from an ocular vascular disease have been previously treated with anti-VEGF treatment (e.g., monotherapy).

26. The method according to the preceding embodiments wherein the ocular vascular disease is DME and the treatment of patients suffering from DME includes a fixed every 8^{th} week (Q8W) dosing schedule following treatment initiation (In one embodiment the treatment initiation includes 5 to 7 monthly administrations; in one embodiment the treatment initiation includes 6 monthly administrations).

27. The method according to the preceding embodiments wherein the ocular vascular disease is DME and the treatment of patients suffering from DME includes a fixed Q12W dosing schedule following treatment initiation (In one embodiment the treatment initiation includes 5 to 7 monthly administrations; in one embodiment the treatment initiation includes 6 monthly administrations).

28. The method according to embodiment 27 wherein, following the treatment initiation, first one dose cycle of Q8W follows before the fixed Q12W dosing schedule.

29. The method according to the preceding embodiments wherein the ocular vascular disease is DME and the treatment of patients suffering from DME includes following treatment initiation a dosing schedule that extends the administration interval in stable absence of disease, or shortens the interval if there is disease activity (In one embodiment the treatment initiation includes 3 to 7 monthly administrations; in one embodiment the treatment initiation includes 4 to 6 monthly administrations).

30. The method according to embodiment 29 wherein such dosing schedule includes that the patient receives Q8W or Q12W or Q16W dosing, dependent on their disease state (in one embodiment Q4W or Q8W or Q12W or Q16W dosing, dependent on their disease state).

31. The method according to embodiment 28 or 29, wherein the stable absence of disease is determined as
Central Subfield Thickness (CST) increased by <50 μm; and/or
Best Corrected Visual Acuity (BCVA/ETDRS) decreased by <5 letters
and the disease activity is determined as
Central Subfield Thickness (CST) increased by ≥50 μm; and/or
Best Corrected Visual Acuity (BCVA/ETDRS) decreased by ≥5 letters.

32. The method according to embodiment 28 or 29, wherein the stable absence of disease is determined as
Central Subfield Thickness (CST) is below about 300 μm (In one embodiment below 325 μm measured by spectral domain optical coherence tomography (SD-OCT) with a Spectralis™ device; in one embodiment below 315 μm measured by spectral domain optical coherence tomography (SD-OCT) with a Cirrus™ device; in one embodiment below 315 μm measured by spectral domain optical coherence tomography (SD-OCT) with a Topcon™ device; in one embodiment below 295 μm measured by spectral domain optical coherence tomography (SD-OCT) with a Optovue™ device),
and the disease activity is determined as
Central Subfield Thickness (CST) is above about 300 μm (In one embodiment above 325 μm measured by spectral domain optical coherence tomography (SD-OCT) with a Spectralis™ device; in one embodiment above 315 μm measured by spectral domain optical coherence tomography (SD-OCT) with a Cirrus™ device; in one embodiment above 315 μm measured by spectral domain optical coherence tomography (SD-OCT) with a Topcon™ device; in one embodiment above 295 μm measured by spectral domain optical coherence tomography (SD-OCT) with a Optovue™ device).

33. The method according to the preceding embodiments wherein the treatment of patients suffering from AMD (in one embodiment wet AMD) includes following treatment initiation (In one embodiment the treatment initiation includes 3 to 7 monthly administrations; in one embodiment the treatment initiation includes 4 to 6 monthly administrations) a dosing schedule that extends the administration interval in stable absence of disease, or shortens the interval if there is disease activity

34. The method according to embodiment 33 wherein such dosing schedule includes that the patient receives Q8W or Q12W or Q16W dosing, dependent on their disease state (in one embodiment Q4W or Q8W or Q12W or Q16W dosing, dependent on their disease state).

35. The method according to embodiment 33 or 34, wherein the stable absence of disease is determined as
Central Subfield Thickness (CST) increased by <50 μm; and/or
Best Corrected Visual Acuity (BCVA/ETDRS) decreased by <5 letters
and the disease activity is determined as
Central Subfield Thickness (CST) increased by ≥50 μm; and/or
Best Corrected Visual Acuity (BCVA/ETDRS) decreased by ≥5 letters.

36. The method according to embodiment 33 or 34, wherein the stable absence of disease is determined as
Central Subfield Thickness (CST) is below about 300 μm (In one embodiment below 325 μm measured by spectral domain optical coherence tomography (SD-OCT) with a Spectralis™ device; in one embodiment below 315 μm measured by spectral domain optical coherence tomography (SD-OCT) with a Cirrus™ device; in one embodiment below 315 μm measured by spectral domain optical coherence tomography (SD-OCT) with a Topcon™ device; in one embodiment below 295 μm measured by spectral domain optical coherence tomography (SD-OCT) with a Optovue™ device),
and the disease activity is determined as
Central Subfield Thickness (CST) is above about 300 μm (In one embodiment above 325 μm measured by spectral domain optical coherence tomography (SD-OCT) with a Spectralis™ device; in one embodiment above 315 μm measured by spectral domain optical coherence tomography (SD-OCT) with a Cirrus™ device; in one embodiment above 315 μm measured by spectral domain optical coherence tomography (SD-OCT) with a Topcon™ device; in one embodiment above 295 μm measured by spectral domain optical coherence tomography (SD-OCT) with a Optovue™ device).

### EXAMPLES

Treatment of Patient Suffering from Vascular Eye Diseases with a Bispecific Antibody that Binds to Human VEGF and Human ANG2

### Example 1A: Efficacy and Durability of Treatment of Patients Suffering from Diabetic Macular Edema (DME)

### Objectives

### Primary Objective

The primary objective of this study were:

To evaluate the efficacy of the bispecific antibody that binds to human VEGF and human ANG2 comprising the amino acid sequences of SEQ ID NO: 17, of SEQ ID NO: 18, of SEQ ID NO: 19, and of SEQ ID NO: 20 (this antibody VEGFang2-0016 and its production is also described in detail in WO2014/009465 which is incorporated by reference) compared with an active comparator in treatment naïve patients with center-involving diabetic macular edema (CI-DME). Designations of this bispecific anti-VEGF/ANG2 antibody herein are RO6867461 or RG7716 or VEGFang2-0016, or faricimab. Vials of sterile, colorless to brownish, preservative-free solution of RO6867461 for IVT administration of either 1.5 mg or 6 mg dose every 4 weeks were used. The concentration of the bispecific antibody was about 120 mg/ml.

### Secondary Objectives

The secondary objectives for this study were as follows:

To investigate pharmacodynamics and anatomical outcomes informing on the mechanism of action of RO6867461

To investigate the formation of plasma anti RO6867461 antibodies

To explore the duration of effect of RO6867461

### Exploratory Objectives

The exploratory objectives for this study were as follows:

To explore the predictive effect of previous IVT anti-VEGF treatment on efficacy of RO6867461

To evaluate the efficacy and safety of RO6867461 compared with the active comparator in patients with CI DME with previous IVT anti-VEGF treatment.

To evaluate RO6867461 effects on plasma levels of markers of angiogenesis and inflammation

To investigate RO6867461 concentration and, if sample volume allows, biomarkers of angiogenesis and inflammation in aqueous humor samples (optional) and vitreous (optional)

To evaluate improvement in diabetic retinopathy (DR) severity score

### Study Design

This was a multiple-center, multiple-dose, randomized, active comparator-controlled, double masked, three parallel group, 36-week study in patients with CI-DME.

The three groups of this study were as follows:
Arm A: 0.3 mg ranibizumab IVT
Arm B: 1.5 mg RO6867461 IVT
Arm C: 6 mg RO6867461 IVT

Only one eye was selected as the study eye. Where both eyes met all eligibility criteria, the eye with the worse BCVA was defined as the study eye. Where both eyes met all eligibility criteria and have the same BCVA letter score at Day 1, study eye selection was at the investigator's discretion.

### Number of Patients

Up to 210 patients were randomized.

Approximately 150 treatment-naïve patients and approximately 60 patients who have been previously treated with IVT anti-VEGF were enrolled in the study.

Approximately 50 treatment-naïve patients were randomized on each arm (1:1:1 randomization scheme) and approximately 30 patients previously treated with IVT anti-VEGF were randomized into arms A and C.

### Target Population

Male and female patients of ≥18 years of age with CI-DME.

### Inclusion/Exclusion Criteria

### Inclusion Criteria

Patients must have met the following criteria for study entry:

### Ocular Criteria for Study Eye:

Macular edema associated with DR defined as macular thickening by spectral domain optical coherence tomography (SD-OCT) involving the center of the macula: central subfield thickness (CST) of ≥325 μm with Spectralis™ (Heidelberg) at screening (where Spectralis™ is not available, the following devices and CST thresholds were acceptable: CST ≥315 μm for Cirrus™, CST ≥315 μm for Topcon, CST ≥295 μm for Optovue™).

Decreased visual acuity attributable primarily to DME, with best corrected visual acuity (BCVA) letter score of 73-24 letters (inclusive) on Early Treatment Diabetic Retinopathy Study (ETDRS)-like charts (20/40-20/320 Snellen equivalent) on Day 1.

Clear ocular media and adequate pupillary dilatation to allow acquisition of good quality retinal images to confirm diagnosis

### General Criteria:

Diagnosis of diabetes mellitus (DM; Type 1 or Type 2), as defined by the World Health Organization and/or American Diabetes Association

Able and willing to provide written informed consent and to comply with the study protocol according to International Conference on Harmonisation (ICH) and local regulations. Alternatively, a legally authorized representative must be able to consent for the patient according to ICH and local regulations.

Age ≥18 years

For women who were not postmenopausal (i.e. ≥12 months of non-therapy-induced amenorrhea, confirmed by FSH, if not on hormone replacement) or surgically sterile (absence of ovaries and/or uterus) agreement to remain abstinent or use combined contraceptive methods that result in a failure rate of <1% per year during the treatment period and at least through 4 weeks after last dose.

Abstinence is only acceptable if it is in line with the preferred and usual lifestyle of the patient. Periodic abstinence (e.g., calendar, ovulation, symptothermal, or postovulation methods) and withdrawal were not acceptable methods of contraception;

Examples of contraceptive methods with an expected failure rate of <1% per year include male sterilization, hormonal implants, proper use of combined oral or injected hormonal contraceptives, and certain intrauterine devices. Alternatively, two methods (e.g., two barrier methods such as a condom and a cervical cap) may be combined to achieve a failure rate of <1% per year, barrier methods must always be supplemented with the use of a spermicide.

For men: agreement to use a barrier method of contraception during the treatment period for at least 4 weeks after the last dose of study drug

Patients must be willing not to participate in any other clinical trial including an investigational medical product (IMP) or device up to completion of the current study.

### Exclusion Criteria

Patients who meet any of the following criteria were excluded from study entry:

### Ocular Criteria for Study Eye:

Any signs of high-risk PDR defined as:
any vitreous or pre-retinal hemorrhage

NVE ≥½ disc area within an area equivalent to the standard mydriatic ETDRS 7-field on clinical examination

NVD ≥⅓ disc area on clinical examination

Any IVT anti-VEGF treatment within 3 months prior to Day 1

Any panretinal photocoagulation (PRP) treatment prior to Day 1

Any macular laser photocoagulation within 3 months prior to Day 1

### History of Vitreoretinal Surgery

Any IVT or periocular corticosteroid treatment within 3 months prior to Day 1. Any history of Iluvien® or Ozurdex® implants prior to Day 1 will not be permitted

Any cataract surgery or treatment for complications of cataract surgery with steroids within 3 months prior to Day 1

### History of Incisional Glaucoma Surgery

Uncontrolled glaucoma (e.g., progressive loss of visual fields or defined as intraocular pressure [IOP]≥25 mmHg despite treatment with anti-glaucoma medication)

### Concurrent Ocular Conditions in the Study Eye:

### History of Rubeosis

Any current or history of ocular disease other than DME that may confound assessment of the macula or affect central vision (e.g., age-related macular degeneration, retinal vein occlusion, uveitis, angioid streaks, histoplasmosis, active or inactive cytomegalovirus, pathological myopia, retinal detachment, macular traction, macular hole, significant cataract)

Any current ocular condition for which, in the opinion of the investigator, visual acuity loss would not improve from resolution of macular edema (e.g., foveal atrophy, pigment abnormalities, dense sub-foveal hard exudates, non-retinal condition)

Any active ocular infection on Day 1

Any active intraocular inflammation (grade trace or above) on Day 1

### Characteristics for Fellow Eye:

Any anti-VEGF treatment within 7 days prior to Day 1

Any retinal condition that, in the opinion of the investigator, might require anti-VEGF treatment within 7 days from Day 1

### General Criteria:

Any systemic anti-VEGF within 6 months prior to Day 1

Any major illness or major surgical procedure within 1 month prior to Day 1

Any febrile illness within 1 week prior to Day 1

Any stroke or myocardial infarction within 12 months prior to Day 1

Uncontrolled blood pressure (BP; defined as systolic >180 mmHg and/or diastolic >100 mmHg while patient at rest). If a patient's initial reading exceeds these values, a second reading may be taken either 30 or more minutes later on the same day or on another day during the screening period. If the patient's BP needs to be controlled by antihypertensive medication, the patient should be taking the same medication continuously for at least 1 month prior to Day 1.

Patients with glycosylated hemoglobin HbA1c>12% at screening

Untreated diabetes mellitus or initiation of oral anti-diabetic medication or insulin within 4 months prior to Day 1 or anticipated change of anti-diabetic medications within the duration of the study

Renal failure requiring renal transplant, hemodialysis, or peritoneal dialysis within 6 months prior to Day 1 or anticipated to require hemodialysis or peritoneal dialysis at any time during the study

History of other disease, metabolic dysfunction, physical examination finding, or clinical laboratory finding giving reasonable suspicion of a condition that contraindicated the use of the IMP or that might affect interpretation of the results of the study or renders the patient at high risk for treatment complications in the opinion of the investigator

For females of childbearing potential, a positive blood pregnancy test

### Lactating Female

Use of systemic corticosteroids within 1 month prior to Day 1

Any known hypersensitivity to active comparator, fluorescein, any ingredient of the formulation used, dilating eye drops, or any anesthetics and microbial drops used

Any other restriction accorded to the use of the active comparator

Any treatment with an IMP in the 3 months prior to Day 1

### Length of Study

The total duration of the study was up to 40 weeks (from screening through study completion) for each enrolled patient as follows:

Screening: up to 4 weeks

Baseline: Day 1

Study treatment administration period: from Day 1 to Week 20

Observational period: From Week 20 up to Week 36

Safety follow up call: During the observational period and 7 days after ranibizumab administration

### End of Study

The end of the study was defined as the date when the last patient last observation (LPLO) occurs. LPLO was expected to occur 36 weeks after the last patient is enrolled.

### Efficacy and Pharmacodynamic Outcome Measures

The primary analysis population was treatment naïve patients. Additional analyses may be performed in the overall population and in patients previously treated with IVT anti-VEGF.

The primary efficacy outcome measure for this study was the mean change in BCVA (ETDRS letters) from baseline at Week 24 in treatment-naïve patients.

### Anatomic Outcome Measures by SD-OCT:

Mean change from baseline in foveal center point thickness at Week 24

Mean change from baseline in mean CST (1 mm diameter) at Week 24

Proportion of patients with resolution of subretinal and intraretinal fluid at Week 24

Anatomic outcome measures by fundus fluorescein angiography (FFA)

Proportion of patients with resolution of leakage at the macula at Week 24

Change from baseline in the size of the foveal avascular zone at Week 24

### Exploratory Outcome Measures

The exploratory outcome measures for this study included but were not limited to the following:

### BCVA:

Difference in mean BCVA change from baseline between the treatment-naïve patients and patients with previous IVT anti-VEGF (differential effect of RO6867461)

### Durability-Related Exploratory Outcome Measures:

Time to increase of CST by ≥50 μm and/or loss of ≥5 letters of BCVA due to DME compared to values at Week 20

Time to retreatment with 0.3 mg ranibizumab after Week 20

### Results

The primary efficacy analyses included all randomized patients, with patients grouped according to the treatment assigned at randomization.

The primary efficacy variable was the BCVA change from baseline to Week 24. The primary efficacy analysis was performed using a Mixed Model for Repeated Measurement (MMRM) model.

### Best Corrected Visual Acuity

BCVA at a starting test distance of 4 meters was measured prior to dilating eyes by a trained and certified VA examiner masked to study drug arm assignment.

BCVA was measured by using the set of three Precision Vision™ or Lighthouse distance acuity charts (modified ETDRS Charts 1, 2, and R). A VA Manual was provided to the investigators. VA examiner and VA examination room certifications were obtained before any VA examinations were performed.

The BCVA examiner was masked to study eye and treatment assignment and will only perform the refraction and BCVA assessment (e.g. Visual Acuity Specification Manual). The BCVA examiner has also been masked to the BCVA letter scores of a patient's previous visits and only knew the patient's refraction data from previous visits. The BCVA examiner was not allowed to perform any other tasks involving direct patient care.

TABLE
Baseline Ocular Characteristics in the Study Eye
Summary of Baseline Ocular Characteristics in Interest in the Study Eye, All Patients,
Treatment Naïve Patients Protocol: BP30099

| | 0.3 mg | 1.5 mg | 6 mg | |
|---|---|---|---|---|
| | Ranibizumab | RO6867461 | RO6867461 | All Patients |
| | (N = 59) | (N = 54) | (N = 55) | (N = 168) |
| Best Corrected Visual Acuity result | | | | |
| n | 58 | 54 | 53 | 165 |
| Mean (SD) | 61.24 (9.87) | 60.94 (11.11) | 60.15 (10.80) | 60.79 (10.53) |
| Median | 64.00 | 63.50 | 63.0 | 63.00 |
| Min-Max | 33.0-73.0 | 35.0-85.0 | 25.0-73.0 | 25.0-85.0 |
| Best Corrected Visual Acuity Category | | | | |
| n | 58 | 54 | 53 | 165 |
| 20/40 or better | 13 (22.4%) | 15 (27.8%) | 11 (20.8%) | 39 (23.6%) |
| 20/200 or worse | 4 (6.9%) | 3 (5.6%) | 3 (5.7%) | 10 (6.1%) |
| Better than 20/200 but worse than 20/40 | 41 (70.7%) | 36 (66.7%) | 39 (73.6%) | 116 (70.3%) |
| Baseline BCVA 20/40 or | | | | |
| better/worse than 20/40 | | | | |
| n | 58 | 54 | 53 | 165 |
| Worse than 20/40 | 45 (77.6%) | 39 (72.2%) | 42 (79.2%) | 126 (76.4%) |
| 20/40 or better | 13 (22.4%) | 15 (27.8%) | 11 (20.8%) | 39 (23.6%) |
| Baseline BCVA 20/200 or | | | | |
| worse/better than 20/200 | | | | |
| n | 58 | 54 | 53 | 165 |
| Better than 20/200 | 54 (93.1%) | 51 (94.4%) | 50 (94.3%) | 155 (93.9%) |
| 20/200 or worse | 4 (6.9%) | 3 (5.6%) | 3 (5.7%) | 10 (6.1%) |
| Central Subfield Thickness | | | | |
| n | 58 | 54 | 53 | 165 |
| Mean (SD) | 490.88 (139.01) | 535.44 (163.13) | 495.57 (132.70) | 506.97 (145.95) |
| Median | 476.00 | 489.00 | 466.00 | 478.00 |
| Min-Max | 316.0-999.0 | 302.0-1000.0 | 234.0-825.0 | 234.0-1000.0 |

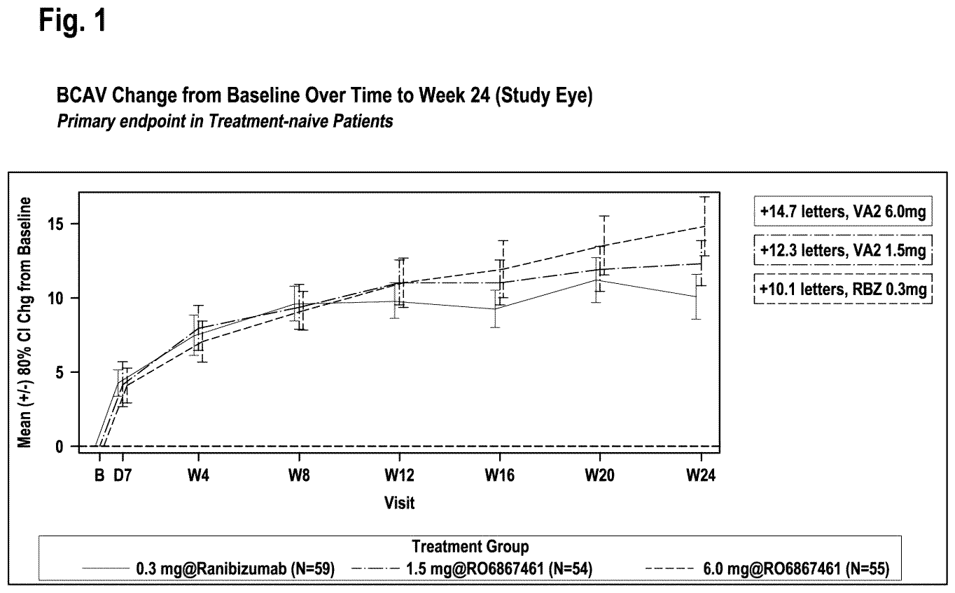

Primary Efficacy Outcome Measure is shown in FIG. 1. The FIG. 1 displays the primary efficacy endpoint: BCVA change from Baseline over Time to Week 24 for so far treatment naive patients. VA2 refers to the bispecific anti-VEGF/ANG2 antibody RO6867461 comprising the amino acid sequences of SEQ ID NO: 17, of SEQ ID NO: 18, of SEQ ID NO: 19, and of SEQ ID NO: 20 (administered intravitreally with a 6.0 mg or 1.5 mg dose), RBZ refers to ranibizumab (Lucentis®) (administered intravitreally with a 0.3 mg dose).

Central Subfield Thickness (CST) Change from Baseline (Study Eye)

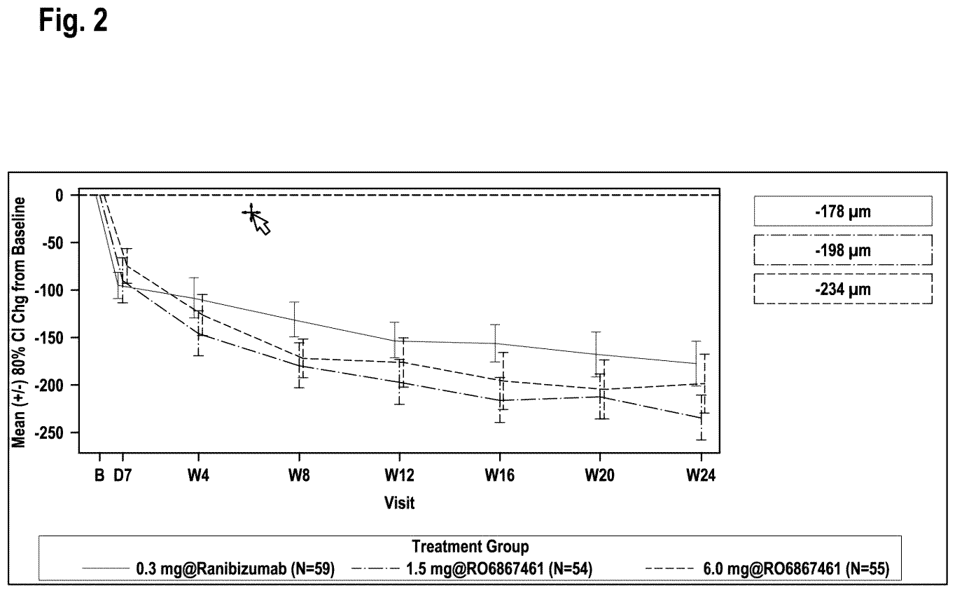

A key secondary endpoint was the change from baseline in CST, central subfield thickness. Results are shown in FIG. 2. The bispecific anti-VEGF/ANG2 antibody RO6867461 comprising the amino acid sequences of SEQ ID NO: 17, of SEQ ID NO: 18, of SEQ ID NO: 19, and of SEQ ID NO: 20 (administered intravitreally with a 6.0 mg or 1.5 mg dose), was compared to ranibizumab (Lucentis®) (administered intravitreally with a 0.3 mg dose). This secondary anatomical endpoint directionally supports BCVA primary outcome.

### Durability/Time to Retreatment

Criteria for Treatment with Ranibizumab During Observational Period

At each visit following the last dose of study treatment (week 20 visit), BCVA was assessed and SD-OCT imaging was performed (except for week 26 visit).

BCVA and CST values obtained at week 24 were compared to those obtained at visit week 20. BCVA and CST values obtained at weeks 28, 32 and 36 were compared to those of week 24.

If the patient met both of the following criteria the patient received a single dose of 0.3 mg ranibizumab and exited the study-:
CST increased by ≥50 μm,
BCVA decreased by ≥5 letters due to DME

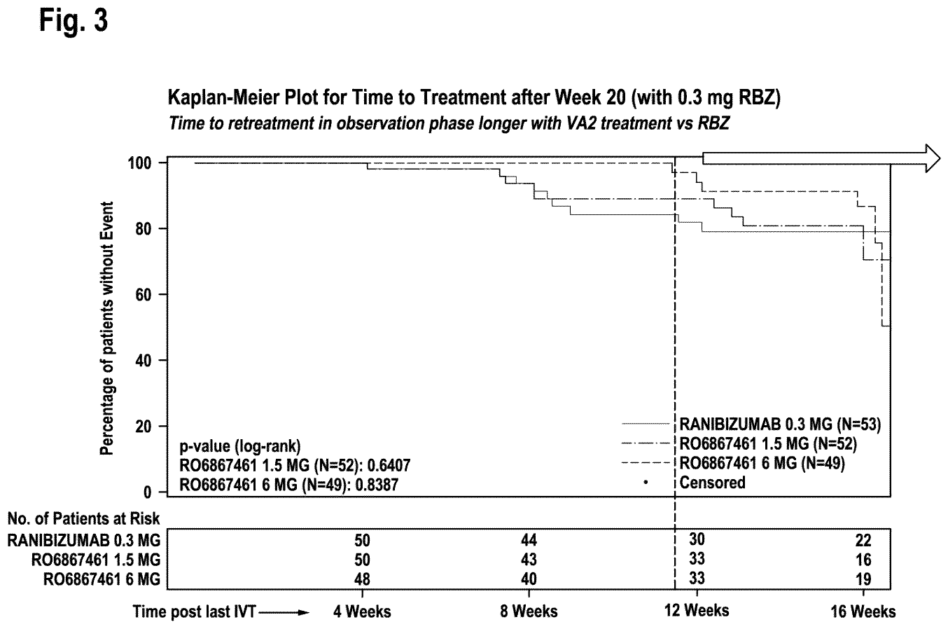

Results are shown in FIG. 3: FIG. 3 shows the time to retreatment after dosing has discontinued (after 20 weeks or 6 monthly doses=Time post last intravitreal (IVT) administration) based on disease activity assessed by both: BCVA decreased by ≥5 letters and CST increased by ≥50 μm. The bispecific anti-VEGF/ANG2 antibody RO6867461 comprising the amino acid sequences of SEQ ID NO: 17, of SEQ ID NO: 18, of SEQ ID NO: 19, and of SEQ ID NO: 20 (administered intravitreally with a 6.0 mg or 1.5 mg dose), was compared to ranibizumab (Lucentis®) (administered intravitreally with a 0.3 mg dose).

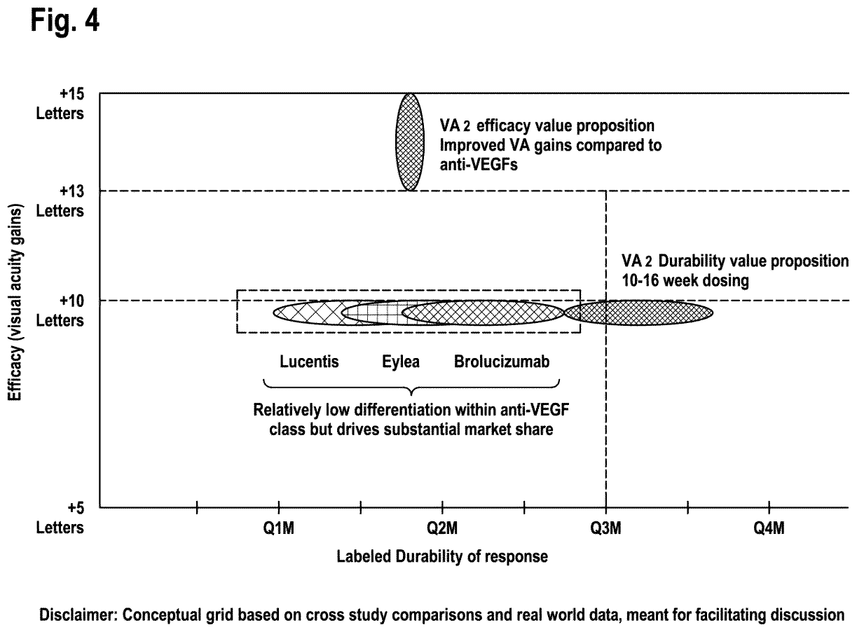

For overview FIG. 4 represents a schematic comparison to other treatment options of DME based on published results (The following agents are compared Lucentis® (ranibizumab), Eylea® (aflibercept), brolucizumab and VA2 (RO6867461/RG7716).

### Example 1B: Efficacy and Durability of Treatment of Patients Suffering from Diabetic Macular Edema (DME)

In a further study analogous to the above described study under Example 1A, patients suffering from DME (e.g., center-involving diabetic macular edema (CI-DME)). are treated with the bispecific antibody that binds to human VEGF and human ANG2 comprising the amino acid sequences of SEQ ID NO: 17, of SEQ ID NO: 18, of SEQ ID NO: 19, and of SEQ ID NO: 20. As active comparator in treatment e.g. aflibercept and/or ranibizumab and/or brolicuzimab will be used. Patients include anti-VEGF treatment-naïve patients (have not been previously treated with anti-VEGF monotherapy with e.g. e.g. aflibercept and/or ranibizumab and/or brolicuzimab)) and also a group of patients which have been previously treated with anti-VEGF monotherapy. Designations of the respective bispecific antibody that binds to human VEGF and human ANG2 are RO6867461 or RG7716. Vials of sterile, colorless to brownish, preservative-free solution of RO6867461 for IVT administration of either 1.5 mg or 6 mg dose are used.

One or more of the following dosing schedules are used:
a) patients suffering from DME will be treated with a fixed Q8W dosing schedule following treatment initiation (e.g. 6 initial monthly injections)
b) patients suffering from DME will be treated with a fixed Q12W dosing (in one schedule with one cycle of Q8W dosing first), following treatment initiation (e.g. 6 initial monthly injections)
c) patients suffering from DME will be treated following treatment initiation (e.g. with 3-7 initial monthly injections) with a dosing regimen that extends the injection interval in stable absence of disease, or shortens the interval if there is disease activity. Such regimen includes e.g. that patient receive Q4W/Q8W/Q12W/Q16W dosing, dependent on their disease state

The disease stability assessment would be based on best-corrected visual acuity (BCVA) and on CST as well as retinal thickness based on Optical coherence tomography (OCT). Outcome measure and results will be evaluated as described e.g. in Example TA. Primary endpoints will be between 45 and 60 weeks.

In one embodiment patients suffering from DME are treatment naïve (have not been previously treated with anti-VEGF monotherapy with e.g. aflibercept and/or ranibizumab and/or brolicuzimab)

In one embodiment patients suffering from DME have been previously treated with anti-VEGF monotherapy with e.g. aflibercept and/or ranibizumab and/or brolicuzimab.

In one embodiment patients suffering from DME will be treated with a fixed Q8W dosing schedule following treatment initiation (e.g. 6 initial monthly injections).

In one embodiment patients suffering from DME will be treated with a fixed Q12W dosing (in one embodiment with one cycle of Q8W dosing first), following treatment initiation (e.g. 6 initial monthly injections).

In one embodiment patients suffering from DME will be treated following treatment initiation (e.g. with 3-7 initial monthly injections) with a dosing regimen that extends the injection interval in stable absence of disease, or shortens the interval if there is disease activity. In one embodiment such regimen includes that patient receive Q4w/Q8w/Q12w/Q16w dosing, dependent on their disease state.

In one embodiment patients suffering from AMD will be treated following treatment initiation (e.g. with 3-4 initial monthly injections) with a dosing regimen that extends the injection interval in stable absence of disease, or shortens the interval if there is disease activity. In one embodiment such regimen includes that patient receive Q4W/Q8W/Q12W/Q16W dosing, dependent on their disease state.

### Example 2A: Efficacy and Durability of Treatment of Patients Suffering from Age-Related Macular Degeneration (AMD)

### Objectives and Endpoints

This study has evaluated the efficacy, safety, and pharmacokinetics of RO6867461 administered at 12- and 16-week intervals in patients with neovascular age-related macular degeneration (nAMD). RO6867461 is a bispecific antibody that binds to human VEGF and human ANG2 comprising the amino acid sequences of SEQ ID NO: 17, of SEQ ID NO: 18, of SEQ ID NO: 19, and of SEQ ID NO: 20 (this antibody VEGFang2-0016 and its production is also described in detail in WO2014/009465 which is incorporated by reference). Designations of this bispecific anti-VEGF/ANG2 antibody herein are RO6867461 or RG7716 or VEGFang2-0016 or faricimab.

Specific objectives and corresponding endpoints for the study are outlined below.

### Objectives and Corresponding Endpoints

### Primary Efficacy Objective

To evaluate the efficacy of RO6867461 on visual acuity when administered at 12- and 16-week intervals

### Corresponding Endpoint

Mean change from baseline BCVA at Week 40 using the ETDRS-like charts Secondary Efficacy Objectives:

1) To evaluate the efficacy of RO6867461 on additional visual acuity outcomes

### Corresponding Endpoints

Mean change from baseline BCVA over time using the ETDRS-like charts
Proportion of patients gaining ≥15, ≥10, ≥5, or ≥0 letters from baseline BCVA over time
Proportion of patients avoiding loss of ≥15, ≥10, ≥5, or ≥0 letters from baseline BCVA over time
Proportion of patients with BCVA of 20/40 or better over time
Proportion of patients with BCVA of 20/200 or worse over time

2) To evaluate the efficacy of RO6867461 on anatomic outcome measures using SD-OCT

### Corresponding Endpoints

Mean change from baseline in CFT over time
Mean change from baseline in mean CST (1 mm diameter) over time
Proportion of patients with intraretinal fluid, subretinal fluid, cysts, or pigment epithelial detachment over time

3) To evaluate the efficacy of RO6867461 on anatomic outcome measures using FFA

### Corresponding Endpoints

Mean change from baseline in total area of CNV at Week 40 and Week 52
Mean change from baseline in total area of CNV component at Week 40 and Week 52
Mean change from baseline in total area of leakage at Week 40 and Week 52

### Exploratory Efficacy Objective

To investigate the incidence of disease activity at Week 24

### Corresponding Endpoints

Proportion of patients with disease activity at Week 24

### Safety Objective

To evaluate the safety of multiple IVT doses of RO6867461 at 12- and 16-week intervals

### Corresponding Endpoints

Incidence and severity of ocular adverse events
Incidence and severity of non-ocular adverse events
Other safety data, including but not limited to, reasons for withdrawal from study, laboratory data, concomitant medications, vital signs, and physical examination results will be listed and summarized descriptively

### Exploratory Pharmacokinetic/Pharmacodynamic Objectives

1) To assess the systemic PK profile of RO6867461

### Corresponding Endpoints

Plasma concentration of RO6867461 at specified timepoints

2) To evaluate the RO6867461, ranibizumab, free VEGF-A, and Ang-2 profile in aqueous humor
Relationship between aqueous humor RO6867461 concentrations or PK parameters and free VEGF-A and Ang-2 concentrations

### Corresponding Endpoints

Relationship between aqueous humor ranibizumab concentrations or PK parameters and free VEGF-A and Ang-2 concentrations
Time course of free VEGF-A and Ang-2 concentrations in aqueous humor

### Immunogenicity Objective

To investigate the formation of plasma anti-RO6867461 antibodies

### Corresponding Endpoints

Incidence of ADAs during the study

### Exploratory Biomarker Objective

To explore levels of potential biomarkers of angiogenesis and inflammation in aqueous humor at baseline and at additional timepoints to assess their response to RO6867461

### Corresponding Endpoints

Relationship between aqueous humor concentration of potential biomarkers with primary and secondary endpoints
   Abbreviations used above:

ADA=anti-drug antibody; Ang-2=angiopoietin-2; BCVA=best corrected visual acuity; CFT=central foveal thickness; CNV=choroidal neovascularization; CST=central subfield thickness; ETDRS=Early Treatment Diabetic Retinopathy Study; FFA=fundus fluorescein angiography; IVT=intravitreal; PK=pharmacokinetic; SD-OCT=spectral domain optical coherence tomography; VEGF-A=vascular endothelial growth factor A.

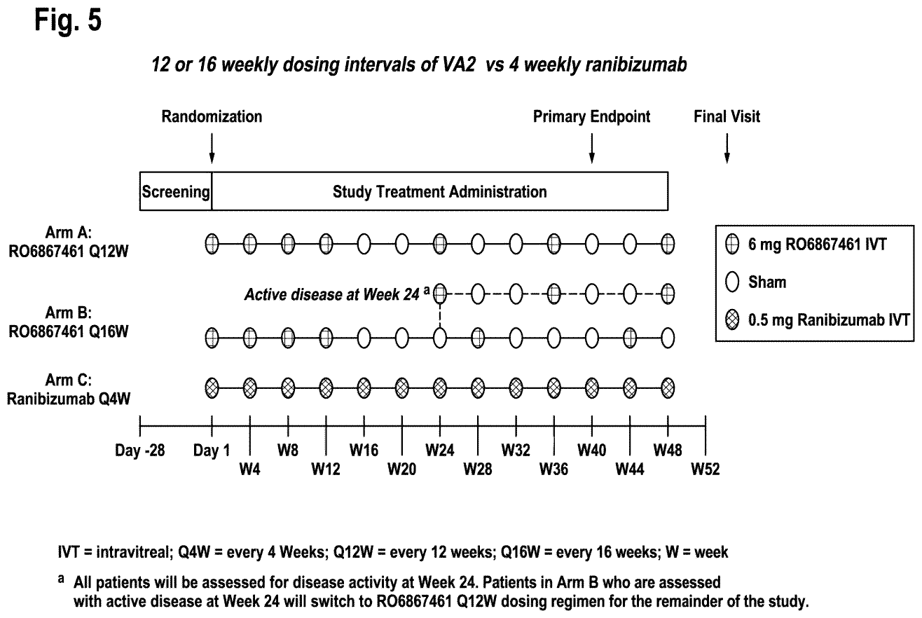

### Study Design (FIG. 5 Presents an Overview of the Study Design)

### Description of Study

This was a Phase II, multicenter, randomized, active comparator-controlled, subject and outcome assessor masked, parallel group, 52-week study to investigate the efficacy, safety, and pharmacokinetics of RO6867461 administered at 12- and 16-week intervals in treatment-naive patients with nAMD.

Approximately 75 patients were enrolled and randomized in a 2:2:1 ratio to one of three treatment arms:
Arm A (Q12W): 6 mg RO6867461 intravitreally (IVT) every 4 weeks up to Week 12 (4 injections), followed by 6 mg RO6867461 IVT every 12 weeks up to Week 48 (injections at Weeks 24, 36, and 48; 3 injections)
Arm B (Q16W): 6 mg RO6867461 IVT every 4 weeks up to Week 12 (4 injections), followed by 6 mg RO6867461 IVT every 16 weeks up to Week 48 (injections at Weeks 28 and 44; 2 injections) A protocol-defined assessment of disease activity at Week 24 requires Arm B patients with active disease (see criteria below) to switch to a 12-weekly dosing regimen of 6 mg RO6867461 for the remainder of the study, with injections commencing at Week 24 and repeated at Weeks 36 and 48.
Arm C (comparator arm): 0.5 mg ranibizumab IVT every 4 weeks for 48 weeks (13 injections) Only one eye will be chosen as the study eye. The total duration of the study for each patient will be up to 56 weeks, divided as follows:
Screening: up to 4 weeks prior to or on the same day as randomization
Randomization: Day 1
Study Treatment Administration: from Day 1 to Week 48
Final Visit: Week 52

Patients have undergone a screening examination within 4 weeks of study treatment administration. The screening and Week 1/Day 1 (randomization) visit may have occurred as a combined visit if all assessments (with the exception of informed consent) were completed within 48 hours. During screening (or the combined screening/Day 1 visit), the patient's eligibility was assessed, including a central review of fundus photography (FP), spectral domain optical coherence tomography (SD-OCT), and fundus fluorescein angiography (FFA) to ensure that CNV secondary to AMD meets the predefined ocular criteria in the study. Patients who were deemed ineligible based on screening results for any of the following reasons were allowed to be re-screened:
Uncontrolled blood pressure
Administrative reason (e.g., unable to schedule Day 1 within 28 days from the screening visit)
Not meeting eligibility criteria for the study eye (in the event the patient might be eligible to participate for the second eye after the initial screening period)

At re-screening, all screening visit assessments were performed (except for FFA imaging collection), provided the Central Reading Center-eligible FFA images were taken within 4 weeks before the new Day 1 visit (randomization).

On Day 1, eligible patients received their first IVT administration of either RO6867461 or ranibizumab according to the randomization schedule described above and following established standard administration procedures. Patients returned to the eye clinic 7 days after their first IVT administration and then every 4 weeks for study treatment administration and assessments as outlined in the schedule of activities in the protocol. Sham IVT administration was delivered to patients randomized to Arms A and B to maintain masking throughout the study period.

All patients were assessed for disease activity at Week 24. Patients randomized to Arm B who had active disease at Week 24 (see criteria below) switched to the Q12W dosing regimen of 6 mg RO6867461 for the remainder of the study, with injections commencing at Week 24 and repeated at Weeks 36 and 48.

Determination of active disease was made if any of the following criteria were met:
Increase in central subfield thickness (CST of >50 μm on Spectralis® OCT compared to average CST over last 2 visits (Weeks 16 and 20)

### Or

Increase in CST of ≥75 μm compared to lowest CST recorded at either Week 16 or Week 20

### Or

Decrease of at least 5 letters of best corrected visual acuity (BCVA) compared with average BCVA over last 2 visits (Weeks 16 and 20) due to nAMD disease activity

### Or

Decrease of ≥10 letters of BCVA compared to highest BCVA recorded at either Week 16 or Week 20 due to nAMD disease activity Or
Presence of new macular hemorrhage due to nAMD activity

Patients will return for a final visit at Week 52. After the final visit, adverse events should be followed up as outlined in the protocol. Assessments performed in case of an unscheduled visit(s) are at the discretion of the investigator

Number of Patients: Approximately 75 treatment-naive patients with nAMD were expected to be enrolled and randomized in this study in the United States.

### Target Population

### Inclusion Criteria

Patients met the following criteria for study entry: Ocular Criteria for Study Eye
Treatment-naive CNV secondary to AMD (nAMD)
Subfoveal CNV or juxtafoveal CNV with a subfoveal component related to the CNV activity by FFA or SD-OCT (as evidenced by subretinal fluid, subretinal hyper-reflective material, evidence of leakage, or hemorrhage)
CNV lesion of all types (predominantly classic, minimally classic, or occult) with: Total lesion size (including blood, atrophy, fibrosis, and neovascularization) of ≤6 disc areas by FFA And CNV component area of ≥50% of total lesion size by FFA And Active CNV confirmed by FFA (evidence of leakage) And CNV exudation confirmed by SD-OCT (presence of fluid)
Clear ocular media and adequate pupillary dilatation to allow acquisition of good quality retinal images to confirm diagnosis General Criteria
Signed Informed Consent Form
Age ≥50 years on Day 1
Ability to comply with the study protocol, in the investigator's judgment
For women of childbearing potential: agreement to remain abstinent (refrain from heterosexual intercourse) or use a contraceptive method with a failure rate of <1% per year during the treatment period and for at least 28 days after the last dose of study treatment
Patients must be willing not to participate in any other clinical trial including an investigational medicinal product (IMP) or device up to completion of the current study

### Exclusion Criteria

Patients who met any of the following criteria were excluded from study entry:

### Ocular Criteria for Study Eye

CNV due to causes other than AMD, such as ocular histoplasmosis, trauma, pathological myopia, angioid streaks, choroidal rupture, or uveitis
Central serous chorioretinopathy at screening
Retinal pigment epithelial tear involving the macula
On FFA Subretinal hemorrhage of >50% of the total lesion area and/or that involves the fovea Fibrosis or atrophy of >50% of the total lesion area and/or that involves the fovea
Any prior or concomitant treatment for CNV including (but not restricted to) IVT treatment (steroids, anti-vascular endothelial growth factor [VEGF], tissue plasminogen activator, ocriplasmin, C3F8 gas, air), periocular pharmacological intervention, argon LASER photocoagulation, verteporfin photodynamic therapy, diode laser, transpupillary thermotherapy, or surgical intervention Cataract surgery within 3 months of baseline assessments (Day 1)
Any other intraocular surgery (pars plana vitrectomy, glaucoma surgery, corneal transplant, radiotherapy)
Prior IVT treatment (including anti-VEGF medication) except for management of cataract complication with steroid IVT treatment Prior periocular pharmacological intervention for other retinal diseases Concurrent Ocular Conditions
Any concurrent intraocular condition in the study eye (e.g., amblyopia, aphakia, retinal detachment, cataract, diabetic retinopathy or maculopathy, or epiretinal membrane with traction) that, in the opinion of the investigator, could either reduce the potential for visual improvement or require medical or surgical intervention during the course of the study
Active intraocular inflammation (grade trace or above) in the study eye on Day 1 (prior to randomization) BCVA letter score of 73 to 24 letters (inclusive) on Early Treatment Diabetic Retinopathy Study (ETDRS)-like charts (20/40 to 20/320 Snellen equivalent) on Day 1
Current vitreous hemorrhage in the study eye
Uncontrolled glaucoma (e.g., progressive loss of visual fields or defined as intraocular pressure [IOP]≥25 mmHg despite treatment with anti-glaucoma medication) in the study eye
Spherical equivalent of refractive error demonstrating more than 8 diopters of myopia in the study eye
History of idiopathic or autoimmune-associated uveitis in either eye
Active infectious conjunctivitis, keratitis, scleritis, or endophthalmitis in either eye on Day 1 (prior to randomization) General Criteria
Any major illness or major surgical procedure within 1 month before screening
Uncontrolled blood pressure ([BP] defined as systolic >180 mmHg and/or diastolic >100 mmHg while patient at rest). If a patient's initial reading exceeds these values, a second reading may be taken later on the same day, or on another day during the screening period. If the patient's BP is controlled by antihypertensive medication, the patient should be taking the same medication continuously for at least 30 days prior to Day 1.
Stroke or myocardial infarction within 3 months prior to Day 1
History of other disease, metabolic dysfunction, physical examination finding, or clinical laboratory findings giving reasonable suspicion of a condition that contraindicated the use of the investigational drug or that might affect interpretation of the results of the study or renders the patient at high risk for treatment complications in the opinion of the investigator
Pregnant or breastfeeding, or intending to become pregnant during the study Women of childbearing potential must have a negative urine pregnancy test result within 28 days prior to initiation of study treatment. If the urine pregnancy test is positive, it must be confirmed by a serum pregnancy test.
Known hypersensitivity to ranibizumab, fluorescein, any ingredients of the formulation used, dilating eye drops, or any of the anesthetic and antimicrobial drops used
Treatment with investigational therapy within 3 months prior to initiation of study treatment

### End of Study

The end of the study was defined as the date when the last patient last visit (LPLV) occurs. LPLV was expected to occur 52 weeks after the last patient is enrolled.

### Length of Study

The total length of the study, from screening of the first patient to the end of the study, was expected to be approximately 18-19 months.

### Investigational Medicinal Products Test Product

RO6867461 Drug Product (120 mg/mL) was provided as a sterile, colorless to brownish liquid and contains no preservatives. Vials of sterile, colorless to brownish, preservative-free solution of RO6867461 for IVT administration of 6 mg dose every were used. The concentration of the bispecific antibody was about 120 mg/ml.

### Dosage and Administration,

### RO6867461, Ranibizumab, and Sham

Patients were given a 50-μL IVT injection of RO6867461 or ranibizumab into the study eye, or a sham administration, according to the randomization schedule as described below
Arm A (Q12W): 6 mg RO6867461 IVT every 4 weeks up to Week 12 (4 injections), followed by 6 mg RO6867461 IVT every 12 weeks up to Week 48 (injections at Weeks 24, 36, and 48; 3 injections)
Arm B (Q16W): 6 mg RO6867461 IVT every 4 weeks up to Week 12 (4 injections), followed by 6 mg RO6867461 IVT every 16 weeks up to Week 48 (injections at Weeks 28 and 44; 2 injections)
Arm C (comparator arm): 0.5 mg ranibizumab IVT every 4 weeks for 48 weeks (13 injections)

Only one eye was chosen as the study eye.

### Study Assessments

At timepoints when several assessments coincide, the following sequence was suggested, at the discretion of the investigator. The order could be adjusted to optimize site personnel and patient's time management, except where explicitly stated as mandatory (i.e., text in italics):
Vital signs
Blood sampling: At visits where FFA is performed, blood sampling and angiography can be performed from the same venous cannula. Blood samples must be collected before angiography.
Ocular assessments and imaging

BCVA: BCVA must have been conducted before pupil dilation. At screening and Day 1 visits, BCVA could be performed before vital signs and blood sampling to avoid unnecessary investigations in those patients who may be a screen failure as a result of BCVA letter score.

Slitlamp examination

Pupil dilation

SD-OCT

FP (+infrared reflectance)

FFA

Dilated binocular indirect high-magnification ophthalmoscopy

IOP: mandatory to be performed after all imaging assessments, and the same method should be used throughout the study period
Aqueous humor sampling (optional)

Disease-Specific Assessments

Unless otherwise noted in schedule of activities (Appendix 1), all ocular assessments were performed for both eyes.

### Best Corrected Visual Acuity

BCVA at a starting test distance of 4 meters was measured prior to dilating eyes by a trained and certified visual acuity (VA) examiner masked to study eye treatment assignment.

BCVA was measured using the set of three Precision Vision™ or Lighthouse distance acuity charts (modified ETDRS Charts 1, 2, and R). A VA Procedure Manual was provided to the investigators. VA examiner and VA examination room certifications were obtained before any VA examinations were performed.

The BCVA examiner was masked to the study eye and treatment assignment and will perform the refraction and BCVA assessments (e.g., VA Specification Manual). The BCVA examiner was also masked to the BCVA letter scores of a patient's previous visits and may only know patient refraction data from previous visits.

### Additional Ocular Assessments

Additional ocular assessments which were performed during the study include the following:
Slitlamp examination (scales for grading flare/cells and vitreous hemorrhage density are detailed in Appendix 2)
Dilated binocular indirect high-magnification ophthalmoscopy
IOP

The method of IOP measurement used for a patient remained consistent throughout the study. IOP measurement of both eyes were performed after all imaging.

At study treatment visits, IOP pressure was conducted prior to study treatment administration and 30 (±15) minutes post-treatment administration in the study eye, and if IOP ≥30 mmHg, IOP should be re-assessed 30 (±15) minutes later. If IOP continued to be elevated, treatment was undertaken at the discretion of the investigator.
Finger count vision assessment

In the study eye, a post-treatment optic nerve head perfusion was assessed for each patient immediately after study treatment administration (maximum within 15 minutes after treatment administration) by testing finger count vision, hand motion, or light perception as appropriate.

### Ocular Imaging

The Central Reading Center provided sites with the Central Reading Center Manual and training materials for study-mandated ocular imaging. Before study images were obtained, site personnel and imaging systems (where applicable) was certified by the reading center as specified in the Central Reading Center Manual. All study subject ocular images were obtained only by trained and Central Reading Center certified personnel on certified/registered equipment at the study sites. A copy of all study subject ocular images were transferred to the central reading center for storage and for independent analysis, including for confirmation of eligibility of defined image-related criteria.

### Week 24 Assessment of Disease Activity

All patients were assessed for disease activity at Week 24. Patients randomized to Arm B who had active disease at Week 24 (see criteria below) switched to the Q12W dosing regimen of 6 mg RO6867461 for the remainder of the study, with injections commencing at Week 24 and repeated at Weeks 36 and 48.

Determination of active disease was made if any of the following criteria are met:
Increase in CST of >50 μm on Spectralis OCT compared to average CST over last 2 visits (Weeks 16 and 20)

### Or

Increase in CST of ≥75 μm compared to lowest CST recorded at either Week 16 or Week 20

### Or

Decrease of at least 5 letters of BCVA compared with average BCVA over last 2 visits (Weeks 16 and 20), due to nAMD disease activity

### Or

Decrease of ≥10 letters of BCVA compared to highest BCVA recorded at either Week 16 or Week 20 due to nAMD disease activity

### Or

Presence of new macular hemorrhage due to nAMD activity

### Results

### Best Corrected Visual Acuity (BCVA) and Durability of BCVA Gains (Time to Retreatment to Maintain BCVA Gain)

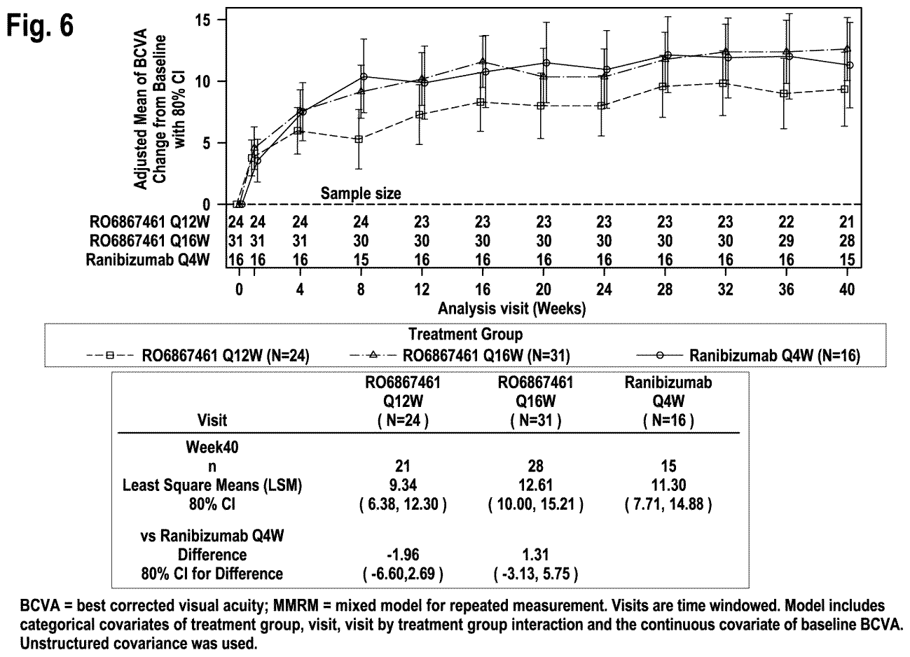

Primary Efficacy Outcome Measure is shown in FIG. 6. The FIG. 6 displays the primary efficacy endpoint: BCVA change from Baseline over Time to Week 40. RO6867461 refers to the bispecific anti-VEGF/ANG2 antibody RO6867461 comprising the amino acid sequences of SEQ ID NO: 17, of SEQ ID NO: 18, of SEQ ID NO: 19, and of SEQ ID NO: 20 (administered intravitreally with a 6.0 mg dose either Q12W or Q16W), ranibizumab (Lucentis®) was administered intravitreally with a 0.3 mg dose Q4W. The initial BCVA gains were fully maintained for the RO6867461 Q12W or Q16W groups and in a similar range as the ranibizumab (Lucentis®) Q4W group.

Central Subfield Thickness (CST) Change from Baseline (Study Eye)

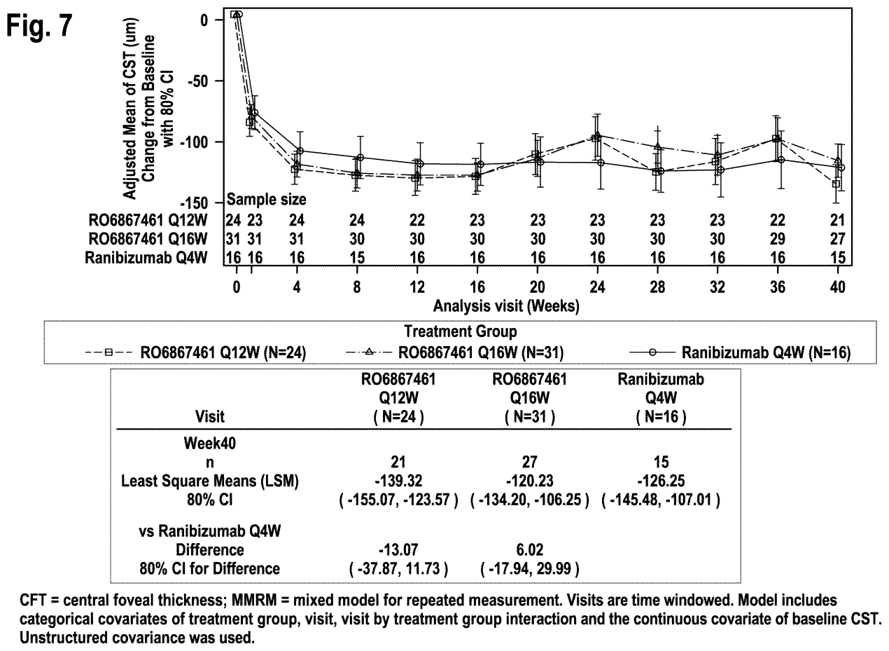

A key secondary endpoint was the change from baseline in CST, central subfield thickness. Results are shown in FIG. 7. The bispecific anti-VEGF/ANG2 antibody RO6867461 comprising the amino acid sequences of SEQ ID NO: 17, of SEQ ID NO: 18, of SEQ ID NO: 19, and of SEQ ID NO: 20 (administered intravitreally with a 6.0 mg dose either Q12W or Q16W), was compared to ranibizumab (Lucentis®) (administered intravitreally with a 0.3 mg dose Q4W). This secondary anatomical endpoint directionally supports BCVA primary outcome There were grater reductions in CST with bispecific anti-VEGF/ANG2 antibody RO6867461 during treatment initiation than with ranibizumab.

### Example 2B: Efficacy and Durability of Treatment of Patients Suffering from Age-Related Macular Degeneration (AMD)

In a further study analogous to the above described study under Example 2A, patients suffering from AMD (e.g. wet age-related macular degeneration (wAMD), especially neovascular AMD) are treated with the bispecific antibody that binds to human VEGF and human ANG2 comprising the amino acid sequences of SEQ ID NO: 17, of SEQ ID NO: 18, of SEQ ID NO: 19, and of SEQ ID NO: 20. As active comparator in treatment e.g., aflibercept and/or ranibizumab and/or brolicuzimab will be used. Patients include anti-VEGF treatment-naïve patients (have not been previously treated with anti-VEGF monotherapy with e.g. aflibercept and/or ranibizumab and/or brolicuzimab) and also a group of patients which have been previously treated with anti-VEGF monotherapy with e.g. aflibercept and/or ranibizumab and/or brolicuzimab. Designations of the respective bispecific antibody that binds to human VEGF and human ANG2 are RO6867461 or RG7716. Vials of sterile, colorless to brownish, preservative-free solution of RO6867461 for IVT administration of either 1.5 mg or 6 mg dose are used.

E.g. the following dosing schedules is used:
Patients suffering from AMD will be treated following treatment initiation (e.g. with 3-7 initial monthly injections) with a dosing regimen that extends the injection interval in stable absence of disease, or shortens the interval if there is disease activity. Such regimen includes e.g. that patient receive Q4W/Q8W/Q12W/Q16W dosing, dependent on their disease state

The disease stability assessment would be based on best-corrected visual acuity (BCVA) and on CST as well as retinal thickness based on Optical coherence tomography (OCT). Outcome measure and results will be evaluated as described e.g. in Example 1A. Primary endpoints will be between 45 and 60 weeks.

### Example 3

### Binding to of the Anti-VEGF/ANG2 Antibody to VEGF, Ang2, FcgammaR and FcRn

VEGF isoforms kinetic affinity including assessment of species-crossreactivity Around 12000 resonance units (RU) of the capturing system (10 μg/ml goat anti human F(ab)′₂; Order Code: 28958325; GE Healthcare Bio-Sciences AB, Sweden) were coupled on a CM5 chip (GE Healthcare BR-1005-30) at pH 5.0 by using an amine coupling kit supplied by the GE Healthcare. The sample and system buffer was PBS-T (10 mM phosphate buffered saline including 0.05% Tween® 20) pH 7.4. The flow cell was set to 25 °C—and the sample block set to 12 °C—and primed with running buffer twice. The bispecific antibody was captured by injecting a 50 nM solution for 30 sec at a flow of 5 μl/min. Association was measured by injection of human hVEGF121, mouse mVEGF120 or rat rVEGF164 in various concentrations in solution for 300 sec at a flow of 30 μl/min starting with 300 nM in 1:3 dilutions. The dissociation phase was monitored for up to 1200 sec and triggered by switching from the sample solution to running buffer. The surface was regenerated by 60 sec washing with a Glycine pH 2.1 solution at a flow rate of 30 μl/min. Bulk refractive index differences were corrected by subtracting the response obtained from a goat anti human F(ab′)₂ surface. Blank injections are also subtracted (=double referencing). For calculation of apparent K_{D} and other kinetic parameters the Langmuir 1:1 model was used. Results are shown in Table 5.

### Ang2 Solution Affinity Including Assessment of Species-Crossreactivity

Solution affinity measures the affinity of an interaction by determining the concentration of free interaction partners in an equilibrium mixture. The solution affinity assay involves the mixing of an <VEGF-ANG-2> bispecific antibody, kept at a constant concentration, with a ligand (=Ang2) at varying concentrations. Maximum possible resonance units (e.g. 17000 resonance units (RU)) of an antibody was immobilized on the CM5 chip (GE Healthcare BR-1005-30) surface at pH 5.0 using an amine coupling kit supplied by the GE Healthcare. The sample and system buffer was HBS-P pH 7.4. Flow cell was set to 25 °C and sample block to 12 °C and primed with running buffer twice. To generate a calibration curve increasing concentrations of Ang2 were injected into a BIAcore™ flowcell containing the immobilized VEGF-ANG-2> bispecific antibody. The amount of bound Ang2 was determined as resonance units (RU) and plotted against the concentration. Solutions of each ligand (11 concentrations from 0 to 200 nM for the VEGF-ANG-2> bispecific antibody) were incubated with 10 nM Ang2 and allowed to reach equilibrium at room temperature. Free Ang2 concentrations were determined from calibration curve generated before and after measuring the response of solutions with known amounts of Ang2. A 4-parameter fit was set with XLfit4 (IDBS Software) using Model 201 using free Ang2 concentration as y-axis and used concentration of antibody for inhibition as x-axis. The affinity was calculated by determining the inflection point of this curve. The surface was regenerated by one time 30 sec washing with a 0.85% H₃PO₄ solution at a flow rate of 30 μl/min. Bulk refractive index differences were corrected by subtracting the response obtained from a blank-coupled surface. Results are shown in Table 6.

### FcRn Steady State Affinity

For FcRn measurement a steady state affinity was used to compare bispecific antibodies against each other. Human FcRn was diluted into coupling buffer (10 μg/ml, Na-Acetate pH5.0) and immobilized on a C1-Chip (GE Healthcare BR-1005-35) by targeted immobilization procedure using a BIAcore™ wizard to a final response of 200 RU. Flow cell was set to 25 °C and sample block to 12 °C and primed with running buffer twice. The sample and system buffer was PBS-T (10 mM phosphate buffered saline including 0.05% Tween® 20) pH 6.0. To assess different IgG concentrations for each antibody, a concentration of 62.5 nM, 125 nM and 250 nM, 500 nM was prepared. Flow rate was set to 30 μl/min and the different samples were injected consecutively onto the chip surface choosing 180 sec association time. The surface was regenerated by injected PBS-T pH 8 for 60 sec at a flow rate of 30 μl/min. Bulk refractive index differences were corrected by subtracting the response obtained from a blank surface. Buffer injections are also subtracted (=double referencing). For calculation of steady state affinity the method from the Bia-Evaluation software was used. Briefly, the RU values (RU max) were plotted against the analysed concentrations, yielding a dose-response curve. Based on a 2-parametric fit, the upper asymptote is calculated, allowing the determination of the half-maximal RU value and hence the affinity. Results are shown in FIG. 5 and Table 7. Analogously the affinity to cyno, mouse and rabbit FcRn can be determined.

### FcgammaRIIIa Measurement

For FcgammaRIIIa measurement a direct binding assay was used. Around 3000 resonance units (RU) of the capturing system (1 μg/ml Penta-His; Qiagen) were coupled on a CM5 chip (GE Healthcare BR-1005-30) at pH 5.0 by using an amine coupling kit supplied by the GE Healthcare. The sample and system buffer was HBS-P+ pH 7.4. The flow cell was set to 25 °C—and sample block to 12 °C—and primed with running buffer twice. The FcgammaRIIIa-His-receptor was captured by injecting a 100 nM solution for 60 sec at a flow of 5 μl/min. Binding was measured by injection of 100 nM of bispecific antibody or monospecific control antibodies (anti-Dig for IgG1 subclass and an IgG4 subclass antibody) for 180 sec at a flow of 30 μl/. The surface was regenerated by 120 sec washing with Glycine pH 2.5 solution at a flow rate of 30 μl/min. Because FcgammaRIIIa binding differs from the Langmuir 1:1 model, only binding/no binding was determined with this assay. In a similar manner FcgammaRIa, and FcgammaRIIa binding can be determined. Results are shown in FIG. 6, where it follows that by introduction of the mutations P329G LALA no more binding to FcgammaRIIIa could be detected.

### Assessment of Independent VEGF- and Ang2-Binding to the <VEGF-ANG-2> Bispecific Antibodies

Around 3500 resonance units (RU) of the capturing system (10 μg/ml goat anti human IgG; GE Healthcare Bio-Sciences AB, Sweden) were coupled on a CM4 chip (GE Healthcare BR-1005-34) at pH 5.0 by using an amine coupling kit supplied by the GE Healthcare. The sample and system buffer was PBS-T (10 mM phosphate buffered saline including 0.05% Tween® 20) pH 7.4. The temperature of the flow cell was set to 25 °C and of the sample block to 12 °C. Before capturing, the flow cell was primed with running buffer twice.

The bispecific antibody was captured by injecting a 10 nM solution for 60 sec at a flow of 5 μl/min. Independent binding of each ligand to the bispecific antibody was analysed by determining the active binding capacity for each ligand, either added sequentially or simultaneously (flow of 30 μl/min):
1. Injection of human VEGF with a concentration of 200 nM for 180 sec (identifies the single binding of the antigen).
2. Injection of human Ang2 with a concentration of 100 nM for 180 sec (identifies single binding of the antigen).
3. Injection of human VEGF with a concentration of 200 nM for 180 sec followed by an additional injection of human Ang2 with a concentration of 100 nM for 180 sec (identifies binding of Ang2 in the presence of VEGF).
4. Injection of human Ang2 with a concentration of 100 nM for 180 sec followed by an additional injection of human VEGF with a concentration of 200 nM (identifies binding of VEGF in the presence of Ang2).
5. Co-Injection of human VEGF with a concentration of 200 nM and of human Ang2 with a concentration of 100 nM for 180 sec (identifies the binding of VEGF and of Ang2 at the same time).

The surface was regenerated by 60 sec washing with a 3 mM MgCl2 solution at a flow rate of 30 μl/min. Bulk refractive index differences were corrected by subtracting the response obtained from a goat anti human IgG surface.

The bispecific antibody is able to bind both antigens mutual independently if the resulting final signal of the approaches 3, 4 & 5 equals or is similar to the sum of the individual final signals of the approaches 1 and 2. Results are shown in the Table below, where VEGFang2-0016 (=RO6867461), is shown to be able to bind mutual independently to VEGF and ANG2

### Assessment of Simultaneous VEGF- and Ang2-Binding to the <VEGF-ANG-2> Bispecific Antibodies

First, around 1600 resonance units (RU) of VEGF (20 μg/ml) were coupled on a CM4 chip (GE Healthcare BR-1005-34) at pH 5.0 by using an amine coupling kit supplied by the GE Healthcare. The sample and system buffer was PBS-T (10 mM phosphate buffered saline including 0.05% Tween® 20) pH 7.4. Flow cell was set to 25 °C and sample block to 12 °C and primed with running buffer twice. Second, 50 nM solution of the bispecific antibody was injected for 180 sec at a flow of 30 μl/min. Third, hAng-2 was injected for 180 sec at a flow of 30 μl/min. The binding response of hAng-2 depends from the amount of the bispecific antibody bound to VEGF and shows simultaneous binding. The surface was regenerated by 60 sec washing with a 0.85% H3PO4 solution at a flow rate of 30 μl/min. Simultaneous binding is shown by an additional specific binding signal of hAng2 to the previous VEGF bound <VEGF-ANG-2> bispecific antibodies.

TABLE
Results: Kinetic affinities to VEGF isoforms from different species

| | VEGFang2-0016-apparent affinity |
|---|---|
| Human VEGF 121 | ≤1 pM (out of Biacore specification) |
| mouseVEGF 120 | no binding |
| Rat VEGF 164 | 14 nM |

TABLE
Results: Solution affinities to Ang2

| | VEGFang2-0016 KD [nM] |
|---|---|
| humanAng2 | 20 |
| cynoAng2 | 13 |
| mouseAng2 | 13 |
| rabbitAng2 | 11 |

TABLE
Results: Affinity to FcRn of <VEGF-ANG-2> bispecific antibodies

| | VEGFang2-0016 [affinity] |
|---|---|
| Human FcRn | no binding |
| Cyno FcRn | no binding |
| Mouse FcRn | no binding |

TABLE
Results Binding to FcgammaRI-IIIa

| | VEGFang2-0016 |
|---|---|
| FcγRIa | No binding |
| FcγRIIa | No binding |
| FcγRIIIa | No binding |

TABLE
Results: Independent binding of VEGF- and Ang2 to <VEGF-ANG-2>
bispecific antibodies

| | | | 3) first | 4) first | 5) |
|---|---|---|---|---|---|
| | | | VEGF | Ang2 | Coinjection |
| | | | then | then | Ang2 + |
| | 1) Ang2 | 2) VEGF | Ang2 | VEGF | VEGF |
| | [RUmax] | [RUmax] | [RUmax] | [RUmax] | [RUmax] |
| VEGFang2- | 174 | 50 | 211 | 211 | 211 |
| 0016 | | | | | |

1-32. (canceled)

33. A method of treating a patient suffering from an ocular vascular disease, the method comprising:
administering to the patient an effective amount of a bispecific antibody which binds to human vascular endothelial growth factor (VEGF) and to human angiopoietin-2 (ANG-2), and comprises the amino acid sequences of SEQ ID NO: 17, of SEQ ID NO: 18, of SEQ ID NO: 19, and of SEQ ID NO: 20,
wherein the bispecific antibody is administered intravitreally every 8 weeks or less frequently in a dose of about 5 to 7 mg,
wherein the ocular vascular disease is Diabetic Macular Edema (DME).

34. The method according to claim 33, wherein the bispecific antibody is administered every 10 to 12 weeks.

35. The method according to claim 33, wherein the bispecific antibody is administered every 11 to 13 weeks.

36. The method according to claim 33, wherein the bispecific antibody is administered every 12 to 14 weeks.

37. The method according to claim 33, wherein the bispecific antibody is administered every 13 to 15 weeks.

38. The method according to claim 33, wherein the bispecific antibody is administered every 14 to 16 weeks.

39. The method according to claim 33, wherein the bispecific antibody is administered in a dose of about 6 mg.

40. The method according to claim 39, wherein the bispecific antibody is administered at a concentration of about 120 mg/ml.

41. The method according to claim 33, wherein the patient has not been previously treated with an anti-VEGF treatment.

42. The method according to claim 33, wherein the patient has been previously treated with an anti-VEGF treatment.

43. The method according to claim 33, wherein the bispecific antibody is administered following a treatment initiation, wherein the treatment initiation comprises 3 to 7 monthly administrations.

44. The method according to claim 43, wherein the treatment includes a fixed every 8^{th} week (Q8W) dosing schedule following the treatment initiation.

45. The method according to claim 43, wherein the treatment includes a fixed Q12W dosing schedule following the treatment initiation.

46. The method according to claim 45, wherein, following the treatment initiation, first one dose cycle of Q8W follows before the fixed Q12W dosing schedule.

47. The method according to claim 43, the treatment of patients suffering from DME further includes, following the treatment initiation, a dosing schedule that extends the administration interval in stable absence of disease, or shortens the interval if there is disease activity.

48. The method according to claim 47 wherein such dosing schedule includes that the patient receives Q8W or Q12W or Q16W dosing, dependent on their disease state.

49. The method according to claim 48 wherein such dosing schedule includes that the patient receives Q8W dosing.

50. The method according to claim 48 wherein such dosing schedule includes that the patient receives Q12W dosing.

51. The method according to claim 48 wherein such dosing schedule includes that the patient receives Q16W dosing.

52. The method according to claim 47, wherein the stable absence of disease is determined as
Central Subfield Thickness (CST) increased by <50 μm; and/or
Best Corrected Visual Acuity (BCVA/ETDRS) decreased by <5 letters and the disease activity is determined as
Central Subfield Thickness (CST) increased by ≥50 μm; and/or
Best Corrected Visual Acuity (BCVA/ETDRS) decreased by ≥5 letters.

53. The method according to claim 33, wherein the effective amount of the bispecific antibody prolongs the time to retreatment and/or prolongs the time to loss of visual acuity and, wherein the retreatment with the bispecific antibody is administered in case of a disease activity which is determined as
Central Subfield Thickness (CST) increase by ≥50 μm; and/or
Best Corrected Visual Acuity (BCVA/ETDRS) decrease by ≥5 letters.
